(19) United States
(12) Patent Application Publication
(10) Pub. No.: US 2017/0359965 A1
(43) Pub. Date: Dec. 21, 2017

(54) POLYLACTIC ACID COMPOSITIONS WITH ACCELERATED DEGRADATION RATE AND INCREASED HEAT STABILITY

(71) Applicant: E I DU PONT DE NEMOURS AND COMPANY, Wilmington, DE (US)

(72) Inventors: KATRINA KRATZ, WILMINGTON, DE (US); BRIAN D. MATHER, SAN DIEGO, CA (US)

(21) Appl. No.: 15/536,339

(22) PCT Filed: Dec. 3, 2015
(86) PCT No.: PCT/US15/63627

§ 371 Date: Jun. 15, 2017

Related U.S. Application Data
(60) Provisional application No. 62/094,199, filed on Dec. 19, 2014.

(51) Int. Cl. A01G 9/10; C08G 63/08; A01H 4/00; C08L 67/04

(52) U.S. Cl. CPC A01G 9/1026; C08L 67/04; C08G 63/08; A01H 4/006; C08L 2201/06; C08L 2203/16

(57) ABSTRACT

The disclosure relates to polylactic acid compositions comprising a degradation additive and a thermal stability additive. The polylactic acid compositions have increased rates of degradation when compared to polylactic acid composition without the disclosed additives. The polylactic acid compositions are particularly suitable for use in artificial seeds.

### FIELD OF THE DISCLOSURE

The present disclosure is directed toward artificial seeds, the production of artificial seeds and degradable components that form a part of the artificial seeds.

### BACKGROUND OF DISCLOSURE

Some plants such as sugarcane, banana, pineapple, citrus, conifers and apple cannot be propagated via seeds due to: a) the loss of genetic identity during reproduction by seed; b) the long duration of growth for the plants before seed production; and c) the poor growth and survival rate of these plants' natural seeds under field growth conditions. Currently, these crops are propagated by either vegetative means or via seedlings. Thus attempts have been made to develop various economical alternatives for their propagation.

Artificial seeds have long been studied as an alternative means to propagate some plants. An artificial seed is an object that is man-made, and which includes components necessary to facilitate plant growth, and from which a plant may grow and be established from its own plant tissue, but wherein the plant tissue is not typically the same as the plant's natural seed. By contrast, a natural seed is produced by plants in a natural biological process without human intervention.

Current artificial seeds are often alginate encapsulated laboratory cultured tissue that can be grown in vitro. These artificial seeds suffer from low survival rates in field environments due to both the encapsulating material as well as biological challenges. Some of the challenges include the desiccation of exposed alginate-encapsulated tissue, attack by soil microorganisms, poor gas exchange of encapsulants, and immaturity and weakness of the laboratory-cultured tissue.

Thus, there remains a need to develop novel and economical methods for improving the viability of the plant tissues incorporated into artificial seeds to enable direct planting of the plantlets into soil.

### SUMMARY OF THE DISCLOSURE

The present disclosure relates to an artificial seed comprising one or more regenerable plant tissues, a container, an airspace, a nutrient source and an additional feature, wherein the additional feature is:
a) a penetrable or degradable region through which the regenerable plant tissue grows;
b) a monolayer portion of the container;
c) a region of the container that flows or creeps between 25° C. and 50° C.;
d) a separable closure which is physically displaced during regenerable plant tissue growth;
e) one or more openings in the sides or bottom of the container;
f) a conical or tapered region leading to an opening less than 2 centimeters wide at the apex and wherein the angle of the conical or tapered region is less than 135 degrees measured from the opposite sides;
g) a plurality of flexible flaps through which the regenerable tissue grows; or
h) a combination thereof; and
   wherein the container comprises a degradable portion comprising a polylactic acid composition and wherein the polylactic acid composition comprises polylactic acid, at least two degradation additives and optionally, a thermal stability additive.

The disclosure also relates to a polylactic acid composition comprising:
1) polylactic acid;
2) at least two degradation additives; and optionally,
3) a thermal stability additive.

The present disclosure also relates to a method comprising the steps of:
A) heating the polylactic acid composition above the softening point of the polylactic acid composition;
B) forming the softened composition into an article; and
C) cooling the formed article,
   wherein the softening temperature of the cooled article is in the range of from 55° C. to 160° C.

The present disclosure also relates to an artificial seed structure comprising a container, an airspace, a nutrient source and one or more additional features listed above as additional features a) through h), wherein at least a portion of the container comprises or consists essentially of a polylactic acid composition, wherein the polylactic acid composition comprises or consists essentially of polylactic acid, at least two degradation additives and a thermal stability additive.

### DETAILED DESCRIPTION

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. In addition, references to the singular may also include the plural (for example, “a” and “an” may refer to one or more) unless the context specifically states otherwise.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

As used herein:

The phrase “regenerable plant tissue” means a tissue capable of regenerating into a mature plant with the same features and genetic identity as the parent plant. Regenerable plant tissues used for encapsulation in artificial seeds as described herein include, but are not limited to, apical or lateral meristematic tissue, callus, somatic embryos, natural embryos, plantlets, leaf whorls, stem and leaf cuttings, natural seeds and buds. A plant of any age can be a source of these tissues. As used herein, “apical meristem” means the meristem at the apical end of the growing stalk. It is the tissue that generates new leaves as well as lateral meristems as the stalk elongates and grows in height.

Various meristematic tissues such as shoot apical meristem, lateral shoot meristem, root apical meristem, vascular meristem and young immature leaves are used in the practice of the present disclosure. In one embodiment, apical shoot meristem tissue can be used. In another embodiment, lateral shoot meristem tissue is used. In another embodiment leaf tissue is used. As used herein, “meristem” encompasses all kinds of meristems available from a plant.

As used herein, “container” means any hollow structure that can hold the regenerable plant tissue. The container can have a variety of shapes and forms, so long as the shape allows the container to hold the plant tissue. For example, the container can be spherical, tubular with circular, conical, cubic, ovoid or any other cross-sectional shape. In one embodiment of the disclosure, the regenerable plant tissue can have a volume of between 0.0001% and 90% of the container volume.

“Multilayer” is defined as a structure possessing more than one layer. “Bilayer” is defined as a structure possessing two layers. At least one of the components of the container comprises the polylactic acid composition comprising polylactic acid, the degradation additive and the thermal stability additive.

One class of regenerable plant tissues of interest is micropropagated plant tissue. Micropropagated tissue is typically grown in a highly hydrated environment, and thus typically lacks features such as full stomatal function and protective morphology such as a cuticle layer. These features are important for the regulation of moisture within the tissue and pose an issue for the survival of these tissues outside of the micropropagation environment. In particular, the field environment can be particularly harsh and challenging for the survival of micropropagated tissues. Micropropagated sugarcane plantlets lack desiccation tolerance and typically exhibit low survival in the field environment. The traditional solution for this is to condition the sugarcane plantlets in a greenhouse, however this is costly and time consuming and results in plants that are too large to plant economically in production fields. In order to support the survival of these tissues in a field environment, it is critical to offer protection from desiccation. This protection may involve protecting the tissue from wind, and creating a humid local environment around the tissue. This can be accomplished by creating a physical barrier or container around the tissue.

Another feature of micropropagated tissue is that it typically lacks robust, lignified structures such as woody stems. These are important to provide stiffness to a mature plant which prevents the plant from damage during winds. Due in part to the lack of such structures, and the sometimes decreased vigor of these tissues compared to natural seeds, it is challenging for micropropagated tissue to escape a container offering maximum protection against moisture loss and desiccation. Micropropagated sugarcane plantlets possess weak, grassy shoots, which are incapable of puncturing commonly-used packaging materials. Thus, it is important to develop mechanisms enabling the escape and proliferation of these tissues from packaging materials.

The containers can help to reduce the rate of water loss the regenerable plant tissue experiences in the field environment, either through transpiration into the atmosphere or conduction and capillary action into the surrounding soil. The container must also allow sufficient gas permeability, to allow the regenerable plant tissue to obtain the gases it needs for photosynthesis and respiration. This can be accomplished by the use of holes in the structure or by tuning the gas permeability or porosity of the container materials. Additionally, it is beneficial that the container allow the passage of some light to the regenerable plant tissue for photosynthesis. Assuming the container protects the regenerable plant tissue adequately to enable survival and growth, the tissue will grow to a size requiring it to escape and shed the container. This allows the roots to proliferate into the soil to reach additional nutrient and water sources, and allows the leaves and shoots to proliferate to increase photosynthesis.

In some embodiments, the disclosure provides containers for the delivery and successful growth of the regenerable plant tissue. In general, the artificial seed will have a top and bottom end, with the regenerable plant tissue positioned such that the shoots grow toward the top end, and the roots grow toward the bottom end. Suitable architectures for the artificial seeds can be found in US 2013/0180173, which is incorporated herein by reference in its entirety.

In some embodiments, the artificial seed comprises a regenerable plant tissue, a container, an airspace, a nutrient source and one or more additional features, wherein at least a portion of the container comprises a polylactic acid composition wherein the polylactic acid composition comprises or consists essentially of polylactic acid, at least two degradation additives and optionally, a thermal stability additive. The one or more additional features may include one or more of the following mechanisms, including all seven, in order to balance the moisture retentive feature of the artificial seed while allowing the eventual escape and proliferation of the micropropagated tissue:
a) In some embodiments, the artificial seed comprises a penetrable or degradable region through which the regenerable plant tissue grows. This portion of the artificial seed can block moisture loss while allowing shoots and roots of the developing plant to puncture them. It is not feasible for the entire container to be composed of a penetrable material, as this can pose problems for handling, storage and planting. Thus a solution proposed herein involves a structure, combining relatively weak, moisture retaining layers with a relatively robust degradable portion;
b) In another embodiment, the artificial seed comprises a monolayer portion of the container or closure;
c) In another embodiment, the artificial seed comprises a region or closure wherein the closure or region flows or creeps at a temperature between 1 to 50° C. or between 25 to 50° C. These temperature ranges are commensurate with typical ambient temperatures experienced in field environments where this artificial seed would be grown;
d) In other embodiments, the artificial seed comprises a separable closure which is physically displaced during regenerable plant tissue growth, for example, caps, lids or fastener structures that can be displaced by the growing plant. In a some embodiments, the caps, lids or fastener structures are displaced by a telescoping action or via the rupture of a weak adhesive joint;
e) In some embodiments, the artificial seed comprises one or more openings in the sides of the container, the bottom closure or both the side of the container and the bottom closure;
f) In another embodiment, the artificial seed comprises conical or tapered regions at the top, at the bottom or both at the top and bottom of the artificial seed, leading to openings which are small relative to the diameter or cross-section of the artificial seed. These tapered regions can guide the shoots of the regenerable plant tissue toward the opening(s) through which they can escape;
g) In another embodiment, the artificial seed comprises one or more flexible flaps through which the regenerable plant tissue grows. The mechanical behavior of the flaps is designed through material choice and geometrical features (thickness, angle relative to emerging shoots) to enable weak plants to deflect and thereby escape the artificial seed.

In other embodiments, the disclosure relates to an artificial seed structure comprising a container, an airspace, a nutrient source, and one or more of the additional features listed above in a) through g), wherein at least a portion of the container comprises a polylactic acid composition and wherein the polylactic acid compiosition comprises or consists essentially of polylactic acid, at least two degradation additives and a thermal stability additive. The addition of regenerable plant tissue to the nutrient source forms the artificial seed.

It is important that the artificial seed is able to provide both an environment that is able to retain moisture (that is, a closed or partially closed structure) and a separable or weak layer that the regenerable plant tissue is able to grow through or separate from the structure in order to develop and mature into the desired plant. One mechanism which is proposed to achieve the balance of moisture retention and plant release is the use of a bi- or multilayer container, wherein the inner wall is water insoluble, and retains moisture, but is weak enough to be punctured by the growing regenerable plant tissue and an outer wall which is the degradable portion but is mechanically robust and protects the artificial seed and regenerable plant tissue therein from mechanical damage.

In other embodiments, the container comprises a weak seam or slotted edge, allowing it to open and release the growing tissue. The weak seam may be created in the container by any means known in the art, including but not limited to perforation, thinning a region of the wall of the container, pre-stressing, creasing, or cracking a region of the container. In one embodiment, the container is an extruded cylindrical tube in which a weak seam is created along one or more edges by extruding a thinner region of material along the seam. In another embodiment, the container is a cylindrical tube with a slot cut along one edge. The material of the container is then flexible enough to allow the plantlet to push the container open. In one embodiment, the container can be constructed of two or more pieces or parts, which may be separable by the growth of the tissue or by dissolution or degradation of an adhesive connecting them. In one embodiment, the container consists of an extruded cylindrical tube with bands of soluble or degradable material along the length of the cylinder. This can be achieved through extrusion of a bi-component or multicomponent, or through the assembly of pieces using adhesive or heat sealing. In another embodiment, the container consists of two longitudinal halves of a tube, which are connected by adhesive. In another embodiment, two halves are connected along one edge through the use of, for example, heat sealing or adhesives, such that a hinged structure is created. In general, any of the known adhesive compositions can be used, provided that the adhesive is suitable for use in a moist environment. In some embodiments, the container consists of two connected sections of a tube. The connected sections may possess different porosity and/or degradability. The sections may be connected by means including, but not limited to, insertion, tape or an adhesive. In one embodiment the top section is composed of plastic and the bottom section is composed of paper.

The container may possess a conical or tapered feature. The angle of the conical feature, measured from one side of the conical section to the opposite side, may be varied, preferably less than 179 degrees, more preferably less than 135 degrees and most preferably less than 100 degrees. A conical tube is defined herein as a cylindrical tube with one or more conical features connected to it. The conical feature may be made of the same material as the cylindrical tube, or a different material. The conical or tapered feature may possess one or more holes, through which the plant can grow. Additionally, the holes provide rapid gas exchange. The size of the holes can vary from 0.1 to 30 millimeters (mm), preferably from 1 to 20 mm and more preferably from 3 to 15 mm.

The container may be expandable or collapsible, such that prior to planting (for instance during storage) the artificial seed occupies a smaller volume than it does after planting. The container may possess an expandable portion or component. As used herein, “expandable” means the capability of increasing in size. This is achieved for instance with concentric tubular or cylindrical containers that can be telescoped to form a longer tube.

As used herein, “telescoping” means the movement of two contacting objects in opposite directions without breaking contact. Also, the container may be partly or completely foldable, such that the folded container, prior to planting, occupies less space than the unfolded container after planting. The container may have pleated or ribbed sections, allowing collapsing while maintaining the same overall shape as the expanded version. The container may expand through the unfolding of an accordion-like structure. The container may possess rigidifying elements. As used herein, “stretching” means the act of elongation through deformation in one or more directions. As used herein, “a rigidifying element” means an element which increases the rigidity of an object. Rigidifying elements include, but are not limited to, creases, folds, inflated compartments, and thick or ribbed regions of the container. The container may be formed from a rolled sheet or tube, such that the structure can unroll or unravel, either at the time of planting or afterward through the growth of the tissue. As used herein, “unraveling” means unrolling of a rolled object without loss of the object's overall shape. The container may possess a collapsible film which can be expanded to form a protective tent around the artificial seed. In one embodiment, the container of the artificial seed may also be stretchable. As used herein, “stretching” means the act of elongation through deformation. In one embodiment, the container may be deflatable and inflatable. The deflation may be achieved through the application of external pressure or through vacuum sealing. Upon rupturing the seal, the container may spontaneously re-inflate. Alternatively, gas pressure may be applied to cause the inflation. In some embodiments, a restraint may be used to keep the container in a compact or collapsed form prior to planting. This restraint includes, but is not limited to, a band or tape, a glue or other fastener.

In some embodiments, the artificial seed possesses a closed bottom end, which can contain the nutrient source and moisture. This closed end can help to prevent the moisture from draining into the surrounding soil. Holes on the sides of the container can be situated to allow root growth, while maintaining the closed nature of the bottom end of the artificial seed.

In some embodiments, the artificial seed can comprise a container that is in the form of a packet or a pouch. The packet may be completely sealed or may possess multiple openings. The packet may be flexible or semi-flexible. Semi-flexible is defined herein as being capable of deformation through an external force, but returning to a shape similar to its original shape after removal of the external force. The packet may possess rigidifying elements. The packet may have shapes including, but not limited to, tubular, cylindrical, rectangular, square or round shapes. The packet may consist of multiple layers, or a single layer. The packet may consist of a bilayer or multilayer film. The packet may possess a water soluble outer layer and a moisture retaining water insoluble inner layer.

The container may be transparent, translucent, or opaque. Transparent means capable of transmitting light so that objects can be seen as if there was no intervening material. Translucent means capable of transmitting light, but causing sufficient distortion so as to prevent perception of distinct images.

The size of the container can vary. However, in some embodiments, the container possesses dimensions in the range of from 0.5 to 5 cm diameter and 1 to 30 cm length and with wall thicknesses ranging from 0.01 to 0.25 centimeter (cm).

The artificial seed comprises a container that can be constructed from various materials, provided that at least a portion of the container comprises a polylactic acid composition wherein the polylactic acid composition comprises or consists essentially of polylactic acid, at least two degradation additives and optionally, a thermal stability additive.

The polylactic acid can be a lactic acid homopolymer or a copolymer thereof. Polylactic acid homopolymers can be produced by the polymerization of any one of the lactic acid isomers, for example, D,L-lactic acid, D-lactic acid, L-lactic acid or any combination thereof. If a lactic acid copolymer is used, then the polylactic acid should contain greater than or equal to 50 mole percent of any one of the above listed lactic acid monomers. In some embodiments, the mole percent of the lactic acid monomer in the copolymer can be greater than or equal to 70 mole percent. In still further embodiments, the mole percentage of the lactic acid monomer can be greater than 80 mole percent, or greater than 90 mole percent. In other embodiments, the polylactic acid is a homopolymer consisting of greater than or equal to 99 mole percent polymerized lactic acid.

If a comonomer is used to produce the polylactic acid copolymer, the comonomer can be other known hydroxy acids, or cyclic esters thereof. Suitable hydroxy acids or cyclic esters can include, for example, glycolic acid, glycolide, tartaric acid, malic acid, mandelic acid, hydroxy-valeric acid, 1-hydroxy-1-cyclohexane carboxylic acid, 2-hydroxy-2-(2-tetrahydrofuranyl) ethanoic acid, 2-hydroxy-2-methylpropionic acid, 2-hydroxy-2-methylbutanoic acid, caprolactone or a combination thereof.

The polylactic acid composition comprises at least two degradation additives and, optionally a thermal stability additive. The inclusion of at least two degradation additives has been found to increase the rate that the container breaks down when exposed to soil and or water. Suitable degradation additives can include, for example, a C8 to C18 carboxylic acid, poly(meth)acrylic acid, anhydrides, maleic anhydride containing polymers, polyvinyl alcohol, polyvinyl pyrrolidone, starch, iron powder, iron (III) carboxylate salt, cobalt carboxylate salt, manganese carboxylate salt, polyethylene glycol, clinoptilolite, zeolite, gypsum, diatomaceous earth, calcium phosphate, calcium carbonate, keratin, silica, alumina, clay, cloisite, montmorillonite or a combination thereof. In some embodiments, the degradation additive can comprise or consist essentially of at least two of C8 to C18 carboxylic acid, starch, phthalic anhydride, iron (III) carboxylate, calcium carbonate or a combination thereof. In still further embodiments, the degradation additive comprises or consists essentially of at least two of C8 to C18 carboxylic acid, starch, calcium carbonate or a combination thereof. In one embodiments, the degradation additive is a combination of a C8 to C18 carboxylic acid and starch, and, in another embodiment, the degradation additive is a combination of a C8 to C18 carboxylic acid and calcium carbonate. In some embodiments, the degradation additive consists of a combination of oleic acid and starch.

The polylactic acid composition can also optionally comprise a thermal stability additive. Typically, polylactic acid compositions have heat deflection (softening point) temperatures that are too low to prevent deformation of the artificial seed after it has been planted in the soil. This can be problematic for artificial seed containers that are exposed to the environment, where the temperatures can reach over 60° C. The addition of one or more thermal stability additives can help the degradable portion of the artificial seed withstand temperatures above 60° C. without deforming. In some embodiments, the thermal stability additive can be a nucleating agent which increases the amount of crystallinity in the polylactic acid composition while in other embodiments, the thermal stability additive can be a reinforcing agent.

The degradable portion is a polylactic acid composition comprising polylactic acid, at least two degradation additives and optionally, a thermal stability additive. In some embodiments, the thermal stability additive can be calcium carbonate, a nucleating agent, a reinforcing fiber, glass fiber, nanocellulose, mica, carbon fiber, sisal, wollastonite, sepiolite, a layered mineral, clay or a combination thereof, natural fiber, polymer fiber, cotton, kenaf, sisal or a combination thereof. In other embodiments, the thermal stability additive is a nucleating agent, sepiolite or a combination thereof. In still further embodiments, the thermal stability additive can be a nucleating agent. Suitable nucleating agents can include, for example, talc, boron nitride, silica, kaolin, clay minerals, titanium oxide, alumina, lauric acid, palm itic acid, stearic acid, behenic acid, benzoic acid, p-tert-butylbenzoic acid, terephthalic acid, terephthalic acid monomethyl ester, isophthalic acid, 12-hydroxystearic acid, alkali (earth) metal salts of organic carboxylic acids, stearic acid amide, erucic acid amide, N-stearyl erucic acid amide, N,N′-ethylenebis(stearamide), ethylenebis-12-hydroxystearic acid amide, hexamethylenebis-10-hydroxystearic acid amide, N,N′-methylenebis(stearamide), methylol stearamide, ethylenebis behenic acid amide, ethylenebis stearic acid amide, ethylenebis lauric acid amide, hexamethylenebis stearic acid amide, butylenebis stearic acid amide, N,N′-distearyladipic acid amide, N,N′-distearylterephthalic acid amide, N,N′-cyclohexanebis(stearamide), N-butyl-N′-stearyl urea, N-propyl-N′-stearyl urea, N-stearyl-N′-stearyl urea, the metal salts of diphenyl phosphate, diphenyl phosphite, sodium bis(4-tert-butylphenyl)phosphate and sodium methylene(2,4-tert-butylphenyl)phosphate, bis(p-methylbenzylidene)sorbitol and bis(p-ethylbenzylidene)sorbitol or a combination thereof.

The polylactic acid composition can comprise in the range of from 50 to 99 percent by weight of polylactic acid, 0.1 to 50 percent by weight of the combination of the at least two degradation additives and optionally, in the range of from 0.1 to 40 percent by weight of the thermal stability additive, wherein all of the percentages by weight are based on the total weight of the polylactic acid composition. In other embodiments, the polylactic acid composition can comprise in the range of from 60 to 90 of the polylactic acid, in the range of from 1 to 40 of the degradation additives and optionally, in the range of from 0.1 to 30 of the thermal stability additive. In still further embodiments, the polylactic acid composition can comprise in the range of from 60 to 90 of the polylactic acid, in the range of from 1 to 40 of the degradation additives and optionally, in the range of from 1 to 15 of the thermal stability additive. All of the percentages by weight are based on the total amount of the polylactic acid composition. In some embodiments, the polylactic acid composition comprises or consists essentially of polylactic acid and in the range of from 25 to 35 percent by weight of starch and in the range of from 2 to 5 percent by weight of a C8 to C18 carboxylic acid. In other embodiments, the polylactic acid composition comprises or consists essentially of polylactic acid, in the range of from 25 to 35 percent by weight of starch and in the range of from 2 to 5 percent by weight of a C8 to C18 carboxylic acid and in the range of from 0.1 to 2 percent by weight of ethylene bis(stearamide). In other embodiments, the polylactic acid composition comprises or consists essentially of polylactic acid, 35 to 45 percent by weight of calcium carbonate and 3 to 7 percent by weight of the C8 to C18 carboxylic acid. In other embodiments, the polylactic acid composition comprises or consists essentially of polylactic acid, 35 to 45 percent by weight of calcium carbonate and 3 to 7 percent by weight of the C8 to C18 carboxylic acid and 0.1 to 2 percent by weight of ethylene bis(stearamide). In other embodiments, the polylactic acid composition comprises or consists essentially of polylactic acid, 12 to 18 percent by weight of sepiolite and 2 to 6 percent by weight of the C8 to C18 carboxylic acid. All percentages by weight are based on the total weight of the polylactic acid composition.

The polylactic acid composition can further comprise one or more additives typically found in polymer compositions. Suitable additives can include, for example, plasticizers, antioxidants, tougheners, colorants, fillers, impact modifiers, processing aids, stabilizers, and flame retardants. Antioxidants can include, for example, hydroquinone, IRGANOX® 1010, and vitamin E. Tougheners include but are not limited to styrenic block copolymers, BIOMAX® Strong, poly(butylene adipate terephthalate), poly(caprolactone), poly(ester urethanes), poly(caprolactone) based polyurethanes, natural rubber, HYTREL®, poly(butylene succinate), poly(butylene succinate adipate), polyethylene oxide, poly(propylene glycol), plasticizers and oils. Colorants include but are not limited to pigments and dyes. Fillers include but are not limited to starch, mica and silica. Impact modifiers include but are not limited to PARALOID™ BPM-520, BIOSTRENGTH® 280, core-shell acrylics, and butadiene rubber. Processing aids include but are not limited to erucamide and stearyl erucamide. Stabilizers include, for example, UV stabilizers, hindered amine light stabilizers, antiozonants and organosulfur compounds. Flame retardants include, for example, aluminum trihydroxide (ATH), magnesium hydroxide (MDH), phosphonate esters, triphenyl phosphate, phosphate esters, ammonium pyrophosphate and melamine polyphosphate.

The container can be formed by extruding the polylactic acid composition or by thermoforming sheets of the polylactic acid composition. In the process of thermoforming sheets of the polylactic acid composition, it has been found that controlling the thermal history of the sheet can also help to increase the thermal stability of the degradable portion of the artificial seed. This is particularly important for the embodiments utilizing nucleating agents as the thermal stability additive(s). Due to the use of chill rollers in sheet extrusion, there is often little crystallinity in the cast sheet. During the thermoforming process, the sheet is heated to soften it and allow deformation in the subsequent molding step. The heating of the sheet allows crystallization to begin, which is the desired intent of utilizing the nucleating agents. However, if the crystallization exceeds a certain limit, the material will not be deformable in the molding step. Controlling the temperatures in the heating zones of the thermoforming process and the duration of heating enables the creation of enough crystallinity to impart the desired thermal stability of the final articles while not interfering with deformation during the forming step.

The cylindrical containers can have flat ends at the top and the bottom. In some embodiments, the bottom end of the container is crenellated. As used herein, “crenellation” means the creation of an irregular edge via the use of tabs of material extending from the edge and indentations into the edge. The size of crenellation can be from 0.65 cm to about 2 cm in length, with 2-6 tabs. In another embodiment, crenellation can be from 0.8 cm to about 1.2 cm in length, with 3-4 tabs.

The artificial seed comprises an airspace within the container. The artificial seed can also contain closures. Closures are defined as lids, caps or objects that cover openings. In one embodiment the closure may be separable from the container. The regenerable plant tissue may be capable of lifting off or shedding the separable closure during its growth. Separable closures include but are not limited to caps, inserts, flat films, dome shaped caps and conical caps. The separable closure may be attached to the container using an adhesive or degradable material. The caps or lids may also be attached by simple physical means including but not limited to insertion or crimping.

Artificial seeds can also comprise one or more of a nutrient source, solid objects such as pieces of cotton, insecticides, fungicides, nematicides, antimicrobial compounds, antibiotics, biocides, herbicides, plant growth regulators, plant growth stimulators, microbes, molluscicides, miticides, acaricides, bird repellants, insect repellants, plant hormones, rodent repellants, fertilizers, hydrogels, superabsorbent polymers, fillers, water or a combination thereof. Biocides include, but are not limited to, hypochlorite, sodium dichloro-s-triazinetrione, Plant Preservative Mixture™, obtained from Plant Cell Technology and trichloro-s-triazinetrione. Molluscicides can include, for example, metaldehyde or methiocarb. Acaricides can include, for example, ivermectin or permethrin. A bird repellent is defined as a substance that repels birds. Bird repellants can include, for example, methyl anthranilate, methiocarb, chlorpyrifos and propiconazole. A rodent repellent is defined as a substance that repels rodents. Rodent repellents can include, for example, thiram and methiocarb. Insect repellents can include, for example, N,N-diethyl-m-toluamide, essential oils and citronella oil. Miticides can include, for example, abamectin and chlorfenapyr. Plant hormones can include, for example, abscisic acid, auxins, cytokinins, ethylene and gibberellins. Plant growth regulators can include, for example, ethephon, and. As used herein, “superabsorbents” means absorbents which absorb water or aqueous solutions resulting in a hydrated gel such that the weight of the gel is 30 times or greater the weight of the dry superabsorbent. Superabsorbent polymers can include, for example, crosslinked poly(sodium acrylate), crosslinked poly(acrylic acid), crosslinked poly(acrylic acid) salts, acrylic acid modified starch and crosslinked copolymers of acrylic acid with poly(ethylene glycol) acrylate, poly(ethylene glycol) methacrylate, poly(ethylene glycol) diacrylate, acrylamide, vinyl acetate, acrylic acid salts, bisacrylamide, N-vinyl pyrrolidone, acrylate esters, methacrylate esters, styrenic monomers, diene monomers or a combination thereof. The superabsorbent may be present in the artificial seed in a dry or swollen state. It may be swollen with water or aqueous solutions, including but not limited to nutrient solutions, fertilizer solutions and antimicrobial solutions. The superabsorbent may also be mixed with soil or other components of the nutrient source. In one embodiment, the superabsorbent may be present in a separate compartment of the regenerable plant tissue. The compartment may be connected or not with the compartment containing the regenerable plant tissue. The compartment may be separated by a screen or mesh from the compartment containing the tissue. Microbes can include, for example beneficial microbes, nitrogen fixing bacteria, rhizobium, fungi, azotobacter, microrhyza, microbes that release cellulases, and microbes that participate in degradation of the container.

As used herein, the phrase “nutrient source” means nutrients which can help sustain and provide for the growth of the plant from the regenerable tissue. Suitable nutrients include, but are not limited to, one or more of water, soil, coconut coir, vermiculite, an artificial growth medium, agar, a plant growth regulator, a plant hormone, a superabsorbent polymer, macronutrients, micronutrients, fertilizers, inorganic salts, (including but not limited to nitrate, ammonium, phosphate, potassium and calcium salts) vitamins, sugars and other carbohydrates, proteins, lipids, Murashige and Skoog (MS) nutrient formula, Hoagland's nutrient formula, Gamborg's B-5 medium, nutrient formula and native and synthetic soils, peat, vinasse or a combination thereof. Macronutrients include but are not limited to nitrate, phosphate and potassium. Micronutrients include but are not limited to cobalt chloride, boric acid, ferrous sulfate and manganese sulfate.

The nutrient source can also contain hormones and plant growth regulators including but not limited to, gibberellic acid, indole acetic acid, naphthalene acetic acid (NAA), ethephon, 6-benzylamino purine (6-ABP), 2,4-dichlorophenoxyacetic acid (2,4-D), and abcissic acid.

The nutrients can be present in an aqueous solution or aqueous gel solution, such as those well known in the art of plant propagation, including but not limited to natural and synthetic gels including: agar, agarose, gellan gum, guar gum, gum arabic, GELRITE™, PHYTAGEL™, superabsorbent polymers, silicate gel, carrageenan, amylose, carboxymethyl-cellulose, dextran, locust bean gum, alginate, xanthan gum, gelatin, pectin, starches, zein, polyacrylamide, polyacrylic acid, poly(ethylene glycol) and crosslinked versions thereof. Silicate gels can be formed, for example, by neutralizing a solution of sodium or potassium silicate with acid. In one embodiment, subsequent washing or soaking steps may be used to remove the excess salts. Optionally, the gel can then be infused with nutrients through soaking or other processes. Alternatively, the silicate gel can be formed from silicic acid, or from other precursors, including but not limited to alkoxysilanes, silyl halides, or silazanes.

The soil suitable for application inside the container where the regenerable plant tissue is to be inserted to grow should be able to provide aeration, water, nutrition, and anchorage to the growing regenerable plant tissue. Various kinds of soil that can be used in the container include synthetic soils like METROMIX® and vermiculite. It can also include natural soils such as sand, silt, loam, peat, and mixtures of these soils. The suitable soil can be present such that the container is at most 99% full. It is beneficial to leave at least 1 mm gap between the moist soil and the top of the container.

The regenerable plant tissue within the container is partially embedded or in contact with the nutrient source and can be partially exposed to the airspace within the container. The term “partially exposed to an airspace”, as used herein, refers to a regenerable plant tissue that is either in contact with or has been partially embedded (i.e., 0 to 90% of the tissue submerged) in the nutrient source present in the container, with the remainder exposed to the airspace within the container. The regenerable plant tissue can also be placed on top of the nutrient source. As used herein, “airspace” means a void in the container that is empty of any solid or liquid material, and filled by atmospheric gasses such as air, for example. An airspace, as defined herein does not include the collective voids in a porous or particulate material.

It is advantageous for the function of the artificial seed that the airspace be free of obstructions that limit the growth of the regenerable plant tissue with exception of the limits of the container wall.

In one embodiment, the artificial seed comprises an enclosure formed by two opposing concave elements, attached together at their intersection. The opposing concave elements or cavities can be formed by thermoforming. Furthermore, the cavities can be multiplexed in a grid type arrangement such as in a horticultural tray. In this embodiment, an upper tray is used to make a top portion of the seed and a lower tray is used to make a bottom portion. The bottom portion contains media and the root and base stem portion of the plantlet. The top portion contains an airspace, in addition to the shoots of the plantlet. In this embodiment, the shape of the cavities differs between the trays used to make the top and bottom portions. The top portion possesses a tapered shape, leading to a single, central hole. This may comprise a frusto-conical shape, or a pyramidal shape. The bottom portion possesses a conical or tapered shape. The bottom of this shape may be flat or conical. The bottom tray will have either one or a plurality of holes which allow the roots of the plantlet to escape as they grow. The cross sectional shape of the tray-based seeds may be circular, square, rectangular, square with rounded corners, hexagonal or any desired cross-section. The gap between the cavities in the trays in this embodiment is between about 0.5-25 mm. The connections between the cavities of the trays may be perforated or otherwise weakened in order to facilitate separation of the seeds during planting. In an assembled seed structure, the two trays may be held together by one or more of the following means: heat sealing, RF sealing, ultrasonic welding, an adhesive or wrapping the joint with a tape or film. Another means of joining the trays is through complementary “snap-together” surface features.

Trays may be fabricated through thermoforming of sheets. Methods of thermoforming include but are not limited to those described in “Technology of Thermoforming” Throne, J. L. 1996. Sheets will be fabricated by methods known in the art, such as extrusion and casting from the melt. The material used to make the sheets will be either resin pellets or powder, or mixtures thereof. Sometimes, the use of multiple types of pellets will be advantaged. The sheet may also consist of multiple layers of material with different compositions. The sheet may have thicknesses ranging from 100 um to 2500 um and may be on a roll or consist of individual square or rectangular formed sheets. The thermoforming process entails heating of the sheet to soften it, placing of the sheet on top of a negative (female) mold surface, deforming of the sheet to match the mold surface, trimming of the sheet, and removal from the mold. The sheet may be heated using methods known in the art, including radiant and convection heaters. The sheet may be gripped mechanically in order to move it through the ovens and onto the mold. In placing the sheet over the mold, a seal will be formed to allow vacuum to be applied. Applying vacuum from inside the mold as well as possibly air pressure and/or a mechanical “plug assist” from above will deform the sheet and shape it to match the mold. The plug assist may also possess holes through which air pressure may be applied. The formed part will then be cooled to harden it and trimmed to remove excess material, with the final part removed from the mold. The mold and plug assist may optionally be heated, or cooled. A release agent may be used to aid in removing the final part from the mold. The release agent may consist of one or more of several compounds known in the art, including silicones. The final parts may be stacked and optionally placed in boxes and palletized.

Another method of forming trays involves natural fiber such as bagasse, bamboo and wheat straw fiber. This involves creating a water-based slurry of the natural fiber called a “pulp”. A mesh screen shaped in the form of the tray is placed in contact with the slurry and vacuum is applied to it. This pulls water through the screen and deposits the fibers on the surface of the screen. The tray is the dried and optionally hot-pressed in a complementary mold prior to removal of the final part. Such processes are known as “molded pulp”, “transfer molded pulp” and “thermoformed fiber”. In one embodiment the molded pulp type trays can be laminated with a polymer film. The polymer film can comprise the polylactic acid composition, a polyester, a polyolefin or other thermoformable polymer.

The regenerable plant tissues can be prepared using various methods well known in the relevant art, such as the method of tissue culture of meristematic tissue described in International Publication Number WO2011/085446, the disclosure of which is herein incorporated by reference. Other possible methods include using plant cuttings, embryos from natural seeds or somatic embryos obtained through somatic embryogenesis. In one embodiment meristems can be excised to form explants and cultured to increase the tissue mass. The term “explant” as used herein, refers to tissues which have been excised from a plant to be used in plant tissue culture.

The regenerable plant tissue may also be genetically modified. This genetic modification includes, but is not limited to, herbicide resistance, disease resistance, drought tolerance, and insect resistance. Genetically modified (also known as transgenic) plants may comprise a single transgenic trait or a stack of one or more transgene polynucleotides with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene with a subsequent gene and co-transformation of genes into a single plant cell.

As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, International Publication Numbers WO 1999/25821, WO 1999/25854, WO 1999/25840, WO 1999/25855 and WO 1999/25853, the disclosures of each of which are herein incorporated by reference.

In some embodiments the polynucleotides encoding the polypeptides, alone or stacked with one or more additional insect resistance traits can be stacked with one or more additional input traits (e.g., herbicide resistance, fungal resistance, virus resistance, stress tolerance, disease resistance, male sterility, stalk strength, or a combination thereof) or output traits (e.g., increased yield, modified starches, improved oil profile, balanced amino acids, high lysine or methionine, increased digestibility, improved fiber quality, drought resistance, or a combination thereof). Thus, the polynucleotide embodiments can be used to provide a complete agronomic package of improved crop quality with the ability to flexibly and cost effectively control any number of agronomic pests.

Transgenes useful for preparing transgenic plants include, but are not limited to, the following:

1. Transgenes Conferring Resistance to Insects or Disease:

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example, Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae), McDowell and Woffenden, (2003) Trends Biotechnol. 21(4):178-83 and Toyoda, et al., (2002) Transgenic Res. 11(6):567-82. A plant resistant to a disease is one that is more resistant to a pathogen as compared to the wild type plant.

(B) Genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162, the disclosures of each of which are herein incorporated by reference.

(C) A polynucleotide encoding an insect-specific hormone or pheromone such as an ecdysteroid and juvenile hormone, a variant thereof, a mimetic based thereon or an antagonist or agonist thereof. See, for example, the disclosure by Hammock, et al., (1990) Nature 344:458, of baculovirus expression of cloned juvenile hormone esterase, an inactivator of juvenile hormone.

(D) A polynucleotide encoding an insect-specific peptide which, upon expression, disrupts the physiology of the affected pest. For example, see the disclosures of, Regan, (1994) J. Biol. Chem. 269:9 (expression cloning yields DNA coding for insect diuretic hormone receptor); Pratt, et al., (1989) Biochem. Biophys. Res. Comm. 163:1243 (an allostatin is identified in Diploptera puntata); Chattopadhyay, et al., (2004) Critical Reviews in Microbiology 30(1):33-54; Zjawiony, (2004) J Nat Prod 67(2):300-310; Carlini and Grossi-de-Sa, (2002) Toxicon 40(11):1515-1539; Ussuf, et al., (2001) Curr Sci. 80(7):847-853 and Vasconcelos and Oliveira, (2004) Toxicon 44(4):385-403. See also, U.S. Pat. No. 5,266,317 to Tomalski, et al., who disclose genes encoding insect-specific toxins.

(E) A polynucleotide encoding an enzyme responsible for a hyperaccumulation of a monoterpene, a sesquiterpene, a steroid, hydroxamic acid, a phenylpropanoid derivative or another non-protein molecule with insecticidal activity.

(F) A polynucleotide encoding an enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application WO 1993/02197 in the name of Scott, et al., which discloses the nucleotide sequence of a callase gene. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase and Kawalleck, et al., (1993) Plant Molec. Biol. 21:673, who provide the nucleotide sequence of the parsley ubi4-2 polyubiquitin gene, and U.S. Pat. Nos. 6,563,020; 7,145,060 and 7,087,810.

(G) A polynucleotide encoding a molecule that stimulates signal transduction. For example, see the disclosure by Botella, et al., (1994) Plant Molec. Biol. 24:757, of nucleotide sequences for mung bean calmodulin cDNA clones, and Griess, et al., (1994) Plant Physiol. 104:1467, who provide the nucleotide sequence of a maize calmodulin cDNA clone.

(H) A polynucleotide encoding a hydrophobic moment peptide. See, PCT Application WO 1995/16776 and U.S. Pat. No. 5,580,852 disclosure of peptide derivatives of Tachyplesin which inhibit fungal plant pathogens) and PCT Application WO 1995/18855 and U.S. Pat. No. 5,607,914 (teaches synthetic antimicrobial peptides that confer disease resistance).

(I) A polynucleotide encoding a membrane permease, a channel former or a channel blocker. For example, see the disclosure by Jaynes, et al., (1993) Plant Sci. 89:43, of heterologous expression of a cecropin-beta lytic peptide analog to render transgenic tobacco plants resistant to Pseudomonas solanacearum.

(J) A gene encoding a viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) A gene encoding an insect-specific antibody or an immunotoxin derived therefrom. Thus, an antibody targeted to a critical metabolic function in the insect gut would inactivate an affected enzyme, killing the insect. Cf. Taylor, et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments).

(L) A gene encoding a virus-specific antibody. See, for example, Tavladoraki, et al., (1993) Nature 366:469, who show that transgenic plants expressing recombinant antibody genes are protected from virus attack.

(M) A polynucleotide encoding a developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436. The cloning and characterization of a gene which encodes a bean endopolygalacturonase-inhibiting protein is described by Toubart, et al., (1992) Plant J. 2:367.

(N) A polynucleotide encoding a developmental-arrestive protein produced in nature by a plant. For example, Logemann, et al., (1992) Bio/Technology 10:305, have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, (1995) Current Biology 5(2), Pieterse and Van Loon, (2004) Curr. Opin. Plant Bio. 7(4):456-64 and Somssich, (2003) Cell 113(7):815-6.

(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2):137-149. Also see, U.S. patent application Ser. Nos. 09/950,933; 11/619,645; 11/657,710; 11/748,994; 11/774,121 and U.S. Pat. Nos. 6,891,085 and 7,306,946. LysM Receptor-like kinases for the perception of chitin fragments as a first step in plant defense response against fungal pathogens (US 2012/0110696).

(Q) Detoxification genes, such as for fumonisin, beauvericin, moniliformin and zearalenone and their structurally related derivatives. For example, see, U.S. Pat. Nos. 5,716,820; 5,792,931; 5,798,255; 5,846,812; 6,083,736; 6,538,177; 6,388,171 and 6,812,380.

(R) A polynucleotide encoding a Cystatin and cysteine proteinase inhibitors. See, U.S. Pat. No. 7,205,453.

(S) Defensin genes. See, WO 2003/000863 and U.S. Pat. Nos. 6,911,577; 6,855,865; 6,777,592 and 7,238,781.

(T) Genes conferring resistance to nematodes. See, e.g., PCT Application WO 1996/30517; PCT Application WO 1993/19181, WO 2003/033651 and Urwin, et al., (1998) Planta 204:472-479, Williamson, (1999) Curr Opin Plant Bio. 2(4):327-31; U.S. Pat. Nos. 6,284,948 and 7,301,069 and miR164 genes (WO 2012/058266).

(U) Genes that confer resistance to Phytophthora Root Rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker, et al., Phytophthora Root Rot Resistance Gene Mapping in Soybean, Plant Genome IV Conference, San Diego, Calif. (1995).

(V) Genes that confer resistance to Brown Stem Rot, such as described in U.S. Pat. No. 5,689,035 and incorporated by reference for this purpose.

(W) Genes that confer resistance to Colletotrichum, such as described in US Patent Application Publication US 2009/0035765 and incorporated by reference for this purpose. This includes the Rcg locus that may be utilized as a single locus conversion.

2. Transgenes that Confer Resistance to a Herbicide:

(A) A polynucleotide encoding resistance to a herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824; U.S. patent application Ser. No. 11/683,737 and International Publication WO 1996/33270.

(B) A polynucleotide encoding a protein for resistance to Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 5,094,945, 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and International Publications EP 1173580; WO 2001/66704; EP 1173581 and EP 1173582, which are incorporated herein by reference for this purpose. Glyphosate resistance is also imparted to plants that express a gene encoding a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase. See, for example, U.S. Pat. Nos. 7,462,481; 7,405,074 and US Patent Application Publication Number US 2008/0234130. A DNA molecule encoding a mutant aroA gene can be obtained under ATCC Accession Number 39256, and the nucleotide sequence of the mutant gene is disclosed in U.S. Pat. No. 4,769,061 to Comai. EP Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin. The nucleotide sequence of a phosphinothricin-acetyl-transferase gene is provided in EP Application Numbers 0 242 246 and 0 242 236 to Leemans, et al.; De Greef, et al., (1989) Bio/Technology 7:61, describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1 and 5,879,903, which are incorporated herein by reference for this purpose. Exemplary genes conferring resistance to phenoxy proprionic acids and cyclohexones, such as sethoxydim and haloxyfop, are the Acc1-S1, Acc1-S2 and Acc1-S3 genes described by Marshall, et al., (1992) Theor. Appl. Genet. 83:435.

(C) A polynucleotide encoding a protein for resistance to herbicide that inhibits photosynthesis, such as a triazine (psbA and gs+genes) and a benzonitrile (nitrilase gene). Przibilla, et al., (1991) Plant Cell 3:169, describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Numbers 53435, 67441 and 67442. Cloning and expression of DNA coding for a glutathione S-transferase is described by Hayes, et al., (1992) Biochem. J. 285:173.

(D) A polynucleotide encoding a protein for resistance to Acetohydroxy acid synthase, which has been found to make plants that express this enzyme resistant to multiple types of herbicides, has been introduced into a variety of plants (see, e.g., Hattori, et al., (1995) Mol Gen Genet. 246:419). Other genes that confer resistance to herbicides include: a gene encoding a chimeric protein of rat cytochrome P4507A1 and yeast NADPH-cytochrome P450 oxidoreductase (Shiota, et al., (1994) Plant Physiol 106:17), genes for glutathione reductase and superoxide dismutase (Aono, et al., (1995) Plant Cell Physiol 36:1687) and genes for various phosphotransferases (Datta, et al., (1992) Plant Mol Biol 20:619).

(E) A polynucleotide encoding resistance to a herbicide targeting Protoporphyrinogen oxidase (protox) which is necessary for the production of chlorophyll. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373 and International Publication WO 2001/12825.

(F) The aad-1 gene (originally from Sphingobium herbicidovorans) encodes the aryloxyalkanoate dioxygenase (AAD-1) protein. The trait confers tolerance to 2,4-dichlorophenoxyacetic acid and aryloxyphenoxypropionate (commonly referred to as “fop” herbicides such as quizalofop) herbicides. The aad-1 gene, itself, for herbicide tolerance in plants was first disclosed in WO 2005/107437 (see also, US 2009/0093366). The aad-12 gene, derived from Delftia acidovorans, which encodes the aryloxyalkanoate dioxygenase (AAD-12) protein that confers tolerance to 2,4-dichlorophenoxyacetic acid and pyridyloxyacetate herbicides by deactivating several herbicides with an aryloxyalkanoate moiety, including phenoxy auxin (e.g., 2,4-D, MCPA), as well as pyridyloxy auxins (e.g., fluroxypyr, triclopyr).

(G) A polynucleotide encoding a herbicide resistant dicamba monooxygenase disclosed in US Patent Application Publication 2003/0135879 for imparting dicamba tolerance;

(H) A polynucleotide molecule encoding bromoxynil nitrilase (Bxn) disclosed in U.S. Pat. No. 4,810,648 for imparting bromoxynil tolerance;

(I) A polynucleotide molecule encoding phytoene (crtl) described in Misawa, et al., (1993) Plant J. 4:833-840 and in Misawa, et al., (1994) Plant J. 6:481-489 for norflurazon tolerance.

3. Transgenes Conferring or Contributing to an Altered Grain Characteristic

(A) Altered fatty acids, for example, by

(1) Down-regulation of stearoyl-ACP to increase stearic acid content of the plant. See, Knultzon, et al., (1992) Proc. Natl. Acad. Sci. USA 89:2624 and WO 1999/64579 (Genes to Alter Lipid Profiles in Corn).

(2) Elevating oleic acid via FAD-2 gene modification and/or decreasing linolenic acid via FAD-3 gene modification (see, U.S. Pat. Nos. 6,063,947; 6,323,392; 6,372,965 and WO 1993/11245).

(3) Altering conjugated linolenic or linoleic acid content, such as in WO 2001/12800.

(4) Altering LEC1, AGP, Dek1, Superal1, mi1 ps, various Ipa genes such as Ipa1, Ipa3, hpt or hggt. For example, see, WO 2002/42424, WO 1998/22604, WO 2003/011015, WO 2002/057439, WO 2003/011015, U.S. Pat. Nos. 6,423,886, 6,197,561, 6,825,397 and US Patent Application Publication Numbers US 2003/0079247, US 2003/0204870 and Rivera-Madrid, et al., (1995) Proc. Natl. Acad. Sci. 92:5620-5624.

(5) Genes encoding delta-8 desaturase for making long-chain polyunsaturated fatty acids (U.S. Pat. No. 8,058,571), delta-9 desaturase for lowering saturated fats (U.S. Pat. No. 8,063,269), Primula Δ6-desaturase for improving omega-3 fatty acid profiles.

(6) Isolated nucleic acids and proteins associated with lipid and sugar metabolism regulation, in particular, lipid metabolism protein (LMP) used in methods of producing transgenic plants and modulating levels of seed storage compounds including lipids, fatty acids, starches or seed storage proteins and use in methods of modulating the seed size, seed number, seed weights, root length and leaf size of plants (EP 2404499).

(7) Altering expression of a High-Level Expression of Sugar-Inducible 2 (HSI2) protein in the plant to increase or decrease expression of HSI2 in the plant. Increasing expression of HSI2 increases oil content while decreasing expression of HSI2 decreases abscisic acid sensitivity and/or increases drought resistance (US Patent Application Publication Number 2012/0066794).

(B) Altered phosphorus content, for example, by the

(1) Introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene.

(2) Modulating a gene that reduces phytate content. In maize, this, for example, could be accomplished, by cloning and then re-introducing DNA associated with one or more of the alleles, such as the LPA alleles, identified in maize mutants characterized by low levels of phytic acid, such as in WO 2005/113778 and/or by altering inositol kinase activity as in WO 2002/059324, US Patent Application Publication Number 2003/0009011, WO 2003/027243, US Patent Application Publication Number 2003/0079247, WO 1999/05298, U.S. Pat. No. 6,197,561, U.S. Pat. No. 6,291,224, U.S. Pat. No. 6,391,348, WO 2002/059324, US Patent Application Publication Number 2003/0079247, WO 1998/45448, WO 1999/55882, WO 2001/04147.

(C) Altered carbohydrates affected, for example, by altering a gene for an enzyme that affects the branching pattern of starch or, a gene altering thioredoxin such as NTR and/or TRX (see, U.S. Pat. No. 6,531,648. which is incorporated by reference for this purpose) and/or a gamma zein knock out or mutant such as cs27 or TUSC27 or en27 (see, U.S. Pat. No. 6,858,778 and US Patent Application Publication Number 2005/0160488, US Patent Application Publication Number 2005/0204418, which are incorporated by reference for this purpose). See, Shiroza, et al., (1988) J. Bacteriol. 170:810 (nucleotide sequence of Streptococcus mutant fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Søgaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 1999/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned herein may also be used to affect starch content and/or composition through the interrelationship of the starch and oil pathways.

(D) Altered antioxidant content or composition, such as alteration of tocopherol or tocotrienols. For example, see, U.S. Pat. No. 6,787,683, US Patent Application Publication Number 2004/0034886 and WO 2000/68393 involving the manipulation of antioxidant levels and WO 2003/082899 through alteration of a homogentisate geranyl geranyl transferase (hggt).

(E) Altered essential seed amino acids. For example, see, U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO 1999/40209 (alteration of amino acid compositions in seeds), WO 1999/29882 (methods for altering amino acid content of proteins), U.S. Pat. No. 5,850,016 (alteration of amino acid compositions in seeds), WO 1998/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO 1998/56935 (plant amino acid biosynthetic enzymes), WO 1998/45458 (engineered seed protein having higher percentage of essential amino acids), WO 1998/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO 1996/01905 (increased threonine), WO 1995/15392 (increased lysine), US Patent Application Publication Number 2003/0163838, US Patent Application Publication Number 2003/0150014, US Patent Application Publication Number 2004/0068767, U.S. Pat. No. 6,803,498, WO 2001/79516.

4. Genes Creating a Site for Site-Specific DNA Integration.

This includes the introduction of FRT sites that may be used in the FLP/FRT system and/or Lox sites that may be used in the Cre/Loxp system. For example, see, Lyznik, et al., (2003) Plant Cell Rep 21:925-932 and WO 1999/25821, which are hereby incorporated by reference. Other systems that may be used include the Gin recombinase of phage Mu (Maeser, et al., (1991) Vicki Chandler, The Maize Handbook ch. 118 (Springer-Verlag 1994), the Pin recombinase of E. coli (Enomoto, et al., 1983) and the R/RS system of the pSRi plasmid (Araki, et al., 1992).

5. Genes Affecting Abiotic Stress Resistance

Including but not limited to flowering, ear and seed development, enhancement of nitrogen utilization efficiency, altered nitrogen responsiveness, drought resistance or tolerance, cold resistance or tolerance and salt resistance or tolerance and increased yield under stress.

(A) For example, see: WO 2000/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 199809521.

(B) WO 199938977 describing genes, including CBF genes and transcription factors effective in mitigating the negative effects of freezing, high salinity and drought on plants, as well as conferring other positive effects on plant phenotype.

(C) US Patent Application Publication Number 2004/0148654 and WO 2001/36596 where abscisic acid is altered in plants resulting in improved plant phenotype such as increased yield and/or increased tolerance to abiotic stress.

(D) WO 2000/006341, WO 2004/090143, U.S. Pat. Nos. 7,531,723 and 6,992,237 where cytokinin expression is modified resulting in plants with increased stress tolerance, such as drought tolerance, and/or increased yield. Also see, WO 2002/02776, WO 2003/052063, JP 2002/281975, U.S. Pat. No. 6,084,153, WO 2001/64898, U.S. Pat. No. 6,177,275 and U.S. Pat. No. 6,107,547 (enhancement of nitrogen utilization and altered nitrogen responsiveness).

(E) For ethylene alteration, see, US Patent Application Publication Number 2004/0128719, US Patent Application Publication Number 2003/0166197 and WO 2000/32761.

(F) For plant transcription factors or transcriptional regulators of abiotic stress, see, e.g., US Patent Application Publication Number 2004/0098764 or US Patent Application Publication Number 2004/0078852.

(G) Genes that increase expression of vacuolar pyrophosphatase such as AVP1 (U.S. Pat. No. 8,058,515) for increased yield; nucleic acid encoding a HSFA4 or a HSFAS (Heat Shock Factor of the class A4 or A5) polypeptides, an oligopeptide transporter protein (OPT4-like) polypeptide; a plastochron2-like (PLA2-like) polypeptide or a Wuschel related homeobox 1-like (WOX1-like) polypeptide (U. Patent Application Publication Number US 2011/0283420).

(H) Down regulation of polynucleotides encoding poly (ADP-ribose) polymerase (PARP) proteins to modulate programmed cell death (U.S. Pat. No. 8,058,510) for increased vigor.

(I) Polynucleotide encoding DTP21 polypeptides for conferring drought resistance (US Patent Application Publication Number US 2011/0277181).

(J) Nucleotide sequences encoding ACC Synthase 3 (ACS3) proteins for modulating development, modulating response to stress, and modulating stress tolerance (US Patent Application Publication Number US 2010/0287669).

(K) Polynucleotides that encode proteins that confer a drought tolerance phenotype (DTP) for conferring drought resistance (WO 2012/058528).

Other genes and transcription factors that affect plant growth and agronomic traits such as yield, flowering, plant growth and/or plant structure, can be introduced or introgressed into plants, see e.g., WO 1997/49811 (LHY), WO 1998/56918 (ESD4), WO 1997/10339 and U.S. Pat. No. 6,573,430 (TFL), U.S. Pat. No. 6,713,663 (FT), WO 1996/14414 (CON), WO 1996/38560, WO 2001/21822 (VRN1), WO 2000/44918 (VRN2), WO 1999/49064 (GI), WO 2000/46358 (FR1), WO 1997/29123, U.S. Pat. No. 6,794,560, U.S. Pat. No. 6,307,126 (GAI), WO 1999/09174 (D8 and Rht) and WO 2004/076638 and WO 2004/031349 (transcription factors).

6. Genes Conferring Increased Yield

(A) A transgenic crop plant transformed by a 1-AminoCyclopropane-1-Carboxylate Deaminase-like Polypeptide (ACCDP) coding nucleic acid, wherein expression of the nucleic acid sequence in the crop plant results in the plant's increased root growth, and/or increased yield, and/or increased tolerance to environmental stress as compared to a wild type variety of the plant (U.S. Pat. No. 8,097,769).

(B) Over-expression of maize zinc finger protein gene (Zm-ZFP1) using a seed preferred promoter has been shown to enhance plant growth, increase kernel number and total kernel weight per plant (US Patent Application Publication Number 2012/0079623).

(C) Constitutive over-expression of maize lateral organ boundaries (LOB) domain protein (Zm-LOBDP1) has been shown to increase kernel number and total kernel weight per plant (US Patent Application Publication Number 2012/0079622).

(D) Enhancing yield-related traits in plants by modulating expression in a plant of a nucleic acid encoding a VIM1 (Variant in Methylation 1)-like polypeptide or a VTC2-like (GDP-L-galactose phosphorylase) polypeptide or a DUF1685 polypeptide or an ARF6-like (Auxin Responsive Factor) polypeptide (WO 2012/038893).

(E) Modulating expression in a plant of a nucleic acid encoding a Ste20-like polypeptide or a homologue thereof gives plants having increased yield relative to control plants (EP 2431472).

7. Gene Silencing

In some embodiments the stacked trait may be in the form of silencing of one or more polynucleotides of interest resulting in suppression of one or more target pest polypeptides. In some embodiments the silencing is achieved through the use of a suppression DNA construct.

In some embodiments one or more polynucleotides encoding the polypeptides or fragments or variants thereof may be stacked with one or more polynucleotides encoding one or more polypeptides having insecticidal activity or agronomic traits as set forth supra and optionally may further include one or more polynucleotides providing for gene silencing of one or more target polynucleotides as discussed infra.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The term “suppression” includes lower, reduce, decline, decrease, inhibit, eliminate and prevent. “Silencing” or “gene silencing” does not specify mechanism and is inclusive, and not limited to, anti-sense, cosuppression, viral-suppression, hairpin suppression, stem-loop suppression, RNAi-based approaches and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50% or any integer between 51% and 100% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target protein.

“Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns or the coding sequence.

“Cosuppression” refers to the production of sense RNA transcripts capable of suppressing the expression of the target protein. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see, Vaucheret, et al., (1998) Plant J. 16:651-659 and Gura, (2000) Nature 404:804-808).

Another variation describes the use of plant viral sequences to direct the suppression of proximal mRNA encoding sequences (PCT Publication WO 1998/36083).

Recent work has described the use of “hairpin” structures that incorporate all or part, of an mRNA encoding sequence in a complementary orientation that results in a potential “stem-loop” structure for the expressed RNA (PCT Publication WO 1999/53050). In this case the stem is formed by polynucleotides corresponding to the gene of interest inserted in either sense or anti-sense orientation with respect to the promoter and the loop is formed by some polynucleotides of the gene of interest, which do not have a complement in the construct. This increases the frequency of cosuppression or silencing in the recovered transgenic plants. For review of hairpin suppression, see, Wesley, et al., (2003) Methods in Molecular Biology, Plant Functional Genomics: Methods and Protocols 236:273-286.

A construct where the stem is formed by at least 30 nucleotides from a gene to be suppressed and the loop is formed by a random nucleotide sequence has also effectively been used for suppression (PCT Publication WO 1999/61632).

The use of poly-T and poly-A sequences to generate the stem in the stem-loop structure has also been described (PCT Publication WO 2002/00894).

Yet another variation includes using synthetic repeats to promote formation of a stem in the stem-loop structure. Transgenic organisms prepared with such recombinant DNA fragments have been shown to have reduced levels of the protein encoded by the nucleotide fragment forming the loop as described in PCT Publication WO 2002/00904.

RNA interference refers to the process of sequence-specific post-transcriptional gene silencing in animals mediated by short interfering RNAs (siRNAs) (Fire, et al., (1998) Nature 391:806). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire, et al., (1999) Trends Genet. 15:358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA of viral genomic RNA. The presence of dsRNA in cells triggers the RNAi response through a mechanism that has yet to be fully characterized.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein, et al., (2001) Nature 409:363). Short interfering RNAs derived from dicer activity are typically about 21 to about 23 nucleotides in length and comprise about 19 base pair duplexes (Elbashir, et al., (2001) Genes Dev. 15:188). Dicer has also been implicated in the excision of 21- and 22-nucleotide small temporal RNAs (stRNAs) from precursor RNA of conserved structure that are implicated in translational control (Hutvagner, et al., (2001) Science 293:834). The RNAi response also features an endonuclease complex, commonly referred to as an RNA-induced silencing complex (RISC), which mediates cleavage of single-stranded RNA having sequence complementarity to the antisense strand of the siRNA duplex. Cleavage of the target RNA takes place in the middle of the region complementary to the antisense strand of the siRNA duplex (Elbashir, et al., (2001) Genes Dev. 15:188). In addition, RNA interference can also involve small RNA (e.g., miRNA) mediated gene silencing, presumably through cellular mechanisms that regulate chromatin structure and thereby prevent transcription of target gene sequences (see, e.g., Allshire, (2002) Science 297:1818-1819; Volpe, et al., (2002) Science 297:1833-1837; Jenuwein, (2002) Science 297:2215-2218 and Hall, et al., (2002) Science 297:2232-2237). As such, miRNA molecules can be used to mediate gene silencing via interaction with RNA transcripts or alternately by interaction with particular gene sequences, wherein such interaction results in gene silencing either at the transcriptional or post-transcriptional level.

Methods and compositions are further provided which allow for an increase in RNAi produced from the silencing element. In such embodiments, the methods and compositions employ a first polynucleotide comprising a silencing element for a target pest sequence operably linked to a promoter active in the plant cell; and, a second polynucleotide comprising a suppressor enhancer element comprising the target pest sequence or an active variant or fragment thereof operably linked to a promoter active in the plant cell. The combined expression of the silencing element with suppressor enhancer element leads to an increased amplification of the inhibitory RNA produced from the silencing element over that achievable with only the expression of the silencing element alone. In addition to the increased amplification of the specific RNAi species itself, the methods and compositions further allow for the production of a diverse population of RNAi species that can enhance the effectiveness of disrupting target gene expression. As such, when the suppressor enhancer element is expressed in a plant cell in combination with the silencing element, the methods and composition can allow for the systemic production of RNAi throughout the plant; the production of greater amounts of RNAi than would be observed with just the silencing element construct alone; and, the improved loading of RNAi into the phloem of the plant, thus providing better control of phloem feeding insects by an RNAi approach. Thus, the various methods and compositions provide improved methods for the delivery of inhibitory RNA to the target organism. See, for example, US Patent Application Publication 2009/0188008.

As used herein, a “suppressor enhancer element” comprises a polynucleotide comprising the target sequence to be suppressed or an active fragment or variant thereof. It is recognize that the suppressor enhancer element need not be identical to the target sequence, but rather, the suppressor enhancer element can comprise a variant of the target sequence, so long as the suppressor enhancer element has sufficient sequence identity to the target sequence to allow for an increased level of the RNAi produced by the silencing element over that achievable with only the expression of the silencing element. Similarly, the suppressor enhancer element can comprise a fragment of the target sequence, wherein the fragment is of sufficient length to allow for an increased level of the RNAi produced by the silencing element over that achievable with only the expression of the silencing element.

It is recognized that multiple suppressor enhancer elements from the same target sequence or from different target sequences or from different regions of the same target sequence can be employed. For example, the suppressor enhancer elements employed can comprise fragments of the target sequence derived from different region of the target sequence (i.e., from the 3′UTR, coding sequence, intron, and/or 5′UTR). Further, the suppressor enhancer element can be contained in an expression cassette, as described elsewhere herein, and in specific embodiments, the suppressor enhancer element is on the same or on a different DNA vector or construct as the silencing element. The suppressor enhancer element can be operably linked to a promoter. It is recognized that the suppressor enhancer element can be expressed constitutively or alternatively, it may be produced in a stage-specific manner employing the various inducible or tissue-preferred or developmentally regulated promoters that are discussed elsewhere herein.

In specific embodiments, employing both a silencing element and the suppressor enhancer element the systemic production of RNAi occurs throughout the entire plant. In further embodiments, the plant or plant parts have an improved loading of RNAi into the phloem of the plant than would be observed with the expression of the silencing element construct alone and, thus provide better control of phloem feeding insects by an RNAi approach. In specific embodiments, the plants, plant parts and plant cells can further be characterized as allowing for the production of a diversity of RNAi species that can enhance the effectiveness of disrupting target gene expression.

In specific embodiments, the combined expression of the silencing element and the suppressor enhancer element increases the concentration of the inhibitory RNA in the plant cell, plant, plant part, plant tissue or phloem over the level that is achieved when the silencing element is expressed alone.

As used herein, an “increased level of inhibitory RNA” comprises any statistically significant increase in the level of RNAi produced in a plant having the combined expression when compared to an appropriate control plant. For example, an increase in the level of RNAi in the plant, plant part or the plant cell can comprise at least about a 1° A, about a 1%-5%, about a 5%-10%, about a 10%-20%, about a 20%-30%, about a 30%-40%, about a 40%-50%, about a 50%-60%, about 60-70%, about 70%-80%, about a 80%-90%, about a 90%-100% or greater increase in the level of RNAi in the plant, plant part, plant cell or phloem when compared to an appropriate control. In other embodiments, the increase in the level of RNAi in the plant, plant part, plant cell or phloem can comprise at least about a 1 fold, about a 1 fold-5 fold, about a 5 fold-10 fold, about a 10 fold-20 fold, about a 20 fold-30 fold, about a 30 fold-40 fold, about a 40 fold-50 fold, about a 50 fold-60 fold, about 60 fold-70 fold, about 70 fold-80 fold, about a 80 fold-90 fold, about a 90 fold-100 fold or greater increase in the level of RNAi in the plant, plant part, plant cell or phloem when compared to an appropriate control. Examples of combined expression of the silencing element with suppressor enhancer element for the control of Stinkbugs and Lygus can be found in US Patent Application Publication 2011/0301223 and US Patent Application Publication 2009/0192117.

Some embodiments relate to down-regulation of expression of target genes in insect pest species by interfering ribonucleic acid (RNA) molecules. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein 527A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein.

“Drought” refers to a decrease in water availability to a plant that, especially when prolonged, can cause damage to the plant or prevent its successful growth (e.g., limiting plant growth or seed yield). “Drought tolerance” is a trait of a plant to survive under drought conditions over prolonged periods of time without exhibiting substantial physiological or physical deterioration. “Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery.

The regenerable plant tissue can be obtained from any plant species, including crops such as, but not limited to: a gram inaceous plant, saccharum spp., saccharum spp. hybrids, sugarcane, miscanthus, switchgrass, energycane, sterile grasses, bamboo, cassava, rice, potato, sweet potato, yam, banana, pineapple, citrus, trees, willow, poplar, mulberry, ficus spp., oil palm, date palm, poaceae, verbena, vanilla, tea, hops, Erianthus spp., intergenic hybrids of Saccharum, Erianthus and Sorghum spp., African violet, date, fig, conifers, apple, guava, mango, maple, plum, pomegranate, papaya, avocado, blackberries, garden strawberry, grapes, canna, cannabis, lemon, orange, grapefruit, tangerine, dayap, maize, wheat, sorghum and cotton. In other embodiments, the regenerable plant tissue can be a genetically modified or a micropropagated or a genetically modified, micropropagated version of any of the listed regenerable plant tissues listed above.

In one embodiment, the regenerable plant tissue used in the artificial seed can be from sugarcane. The regenerable plant tissue can be prepared using several methods including excision of meristems from the top of the sugarcane stalks, followed by tissue culture on solid or liquid media, or temporarily immersed in liquid nutrients and combinations thereof. In one embodiment, the regenerable sugarcane tissue can be prepared using tissue culture on a solid medium, followed by temporary immersion in liquid nutrient media.

The meristem tissue can be allowed to grow and proliferate using a proliferation medium. The proliferation medium can include, but is not limited to, culturing in various liquid nutrient media, culturing on solid media, temporary immersion in liquid nutrient media, and any variations thereof. In one embodiment, the proliferation medium used in the current method comprises MS nutrients and can additionally comprise ingredients not limited to: 30 g/L sucrose, one or more cytokinins, including 6-BAP, auxins, or combinations of cytokinin and auxin, with or without inhibitors of the plant hormone, gibberellin. However, other nutrient formulations such as the well-known in the art Gamborg's B-5 medium, other carbon sources such as glucose and mannitol, other cytokinins, such as kinetin and zeatin can also be used.

The meristem tissues can be allowed to proliferate from about 3 weeks to about 52 weeks. The temperature used for proliferation can vary from about 15° C. to about 45° C. Temperature control for growth of the regenerable plant tissues can be achieved using constant temperature incubators as is well known in the relevant art.

Following growth of the meristem tissue, proliferated buds are formed which contain independent meristem structures capable of differentiating into shoots, and subsequently into well-formed plantlets at later stages. As used herein, “proliferated bud tissue” means a meristematic tissue with the capacity to multiply and self-regenerate into similar meristem structures. Over time, the base of this tissue, which was the original plant tissue, can blacken due to polyphenol production and can be removed by mechanical trimming methods well known in the relevant art.

During the steps described above, the meristem tissue can be subjected to light to allow for growth. The light intensity can be from 1 micro (p) Einstein per square meter per second (μE/m²/s) to about 1500 (μE/m²/s). The light can be produced by various devices suitable for this purpose such as fluorescent bulbs, incandescent bulbs, the sun, plant growth bulbs and light emitting diodes (LEDs). The amount of light required for growth of the meristem tissue can vary from 1 hour photoperiod to 24 hours photoperiod. In an embodiment, a 16 hours photoperiod using 30 μE/m²/s can be used.

After the meristem tissue forms the proliferated bud tissue, it can then be cut into small pieces (fragmented) to form tissue fragments. These tissue fragments can be 0.5-10 mm in size. Alternatively, they can be 1-5 mm in size. These tissue fragments can then be cultured to form the regenerable plant tissue, which are suitable for encapsulation in the artificial seeds. The culturing processes can include, but are not limited to, culturing in various liquid nutrient media, culturing on solid media, temporary immersion in liquid culture, and any variations thereof. The regenerable plant tissue that are formed in these processes possess shoots, with or without roots.

The regenerable plant tissue may be partially embedded into the nutrient-source at the bottom of the container of the artificial seed such that part of the tissue is exposed to the airspace above the nutrient source. The regenerable plant tissue can be oriented or not, and can be trimmed to fit inside the container. Alternately, the regenerable plant tissue can be placed in a soil layer in the container, such that airspace is present above it.

The purpose of the airspace is to allow gas exchange with the regenerable plant tissue, helping to sustain the tissue and allow it to grow. The container can possess porosity which can allow a rate of gas transport such that equilibrium can be maintained between the airspace and the outside environment. Thus, as the regenerable plant tissue consumes or releases oxygen or carbon dioxide, due to either respiration or photosynthesis, these gases are rapidly equilibrated with the outside atmosphere. In addition, the exposure of the regenerable plant tissue to the airspace fosters the development of tissue that is better adapted to the harsher conditions the regenerable plant tissue can be exposed to once it emerges from the artificial seed (for example reduced humidity, wind, higher light). In the artificial seed, the regenerable plant tissue is exposed to less harsh conditions due to the protection of the container. The airspace can also provide some thermal insulation for the regenerable plant tissue. In some embodiments, the airspace may consist of multiple compartments. These compartments may be connected or adjoined and may be in communication with each other. The airspace inside the container artificial seed is at least 1% of the total volume of the container.

After assembly of the artificial seed, there may be a period of storage prior to planting seeds in a field. The length of storage varies and may depend on weather conditions, production, and demand. The range of storage times include 0-8 weeks, extending up to as much as 52 weeks. In one embodiment, the artificial seeds are stored in a controlled environment where the regenerable plant tissue does not grow, yet maintains viability for field planting. This approach comprises the use of a controlled environment with temperatures between about 5-15° C. and light between about 0-250 μE.

A second approach uses the storage period to harden and acclimate the regenerable plant tissue using representative growing conditions. For this approach a growth chamber, greenhouse, or screenhouse could be used where the temperatures range from 20-35° C. and light ranges between 5 μE to that of natural sunlight. The regenerable plant tissue may be stored as bare plants or in any of the seed structures included in US Patent Application publication number 2013/0174483, which is incorporated by reference herein.

Any seed structure listed in US Patent Application publication number 2013/0174483, or described herein, may also be used. A greenhouse or a screenhouse may also be used to harden the plants instead of placing them in a growth chamber under sterile conditions.

To prevent fungal contamination of the artificial seed, the container can be treated with a solution of a fungicide prior to its assembly. Many fungicides can be used for this purpose. Examples include, but are not limited to: MAXIM® XL, MAXIM® 4FS, RIDOMIL GOLD®, UNIFORM®, QUILT®, amphotericin B, cycloheximide, nystatin, griseofulvin, pentachloronitrobenzene, thiabendazole, benomyl, 2-(thiocyanatomethylthio)-1,3-benzothiazole, carbendazim, fuberidazole, thiophanate, thiophanate-methyl, chlozolinate, iprodione, procymidone, vinclozolin, imazalil, oxpoconazole, pefurazoate, prochloraz, triflumizole, triforine, pyrifenox, fenarimol, nuarimol, azaconazole, bitertanol, bromuconazole, cyproconazole, difenoconazole, diniconazole, epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole, benalaxyl, furalaxyl, metalaxyl, metalaxyl-M (mefenoxam), oxadixyl, ofurace, aldimorph, dodemorph, fenpropimorph, tridemorph, fenpropidin, piperalin, spiroxamine, edifenphos, iprobenfos, (IBP), pyrazophos, isoprothiolane, benodanil, flutolanil, mepronil, fenfuram, carboxin, oxycarboxin, thifluzamide, furametpyr, penthiopyrad, boscalid, bupirimate, dimethirimol, ethirimol, cyprodinil, mepanipyrim, pyrimethanil, diethofencarb, azoxystrobin, strobilurins, enestrobin, picoxystrobin, pyraclostrobin, kresoxim-methyl, trifloxystrobin, dimoxystrobin, metominostrobin, orysastrobin, famoxadone, fluoxastrobin, fenamidone, pyribencarb, fenpiclonil, fludioxonil, quinoxyfen, biphenyl, chloroneb, dicloran, quintozene (PCNB), tecnazene (TCNB), tolclofos-methyl, etridiazole, ethazole, fthalide, pyroquilon, tricyclazole, carpropamid, diclocymet, fenoxanil, fenhexamid, pyributicarb, naftifine, terbinafine, polyoxin, pencycuron, cyazofam id, am isulbrom, zoxamide, blasticidin-S, kasugamycin, streptomycin, streptomycin sulfate, validamycin, cymoxanil, iodocarb, propamocarb, prothiocarb, binapacryl, dinocap, ferimzone, fluazinam, fentin acetate, fentin chloride, fentin hydroxide, oxolinic acid, hymexazole, octhilinone, fosetyl-Al, phosphorous acid and salts, teclofthalam, triazoxide, flusulfamide, diclomezine, silthiofam, diflumetorim, dimethomorph, flumorph, benthiavalicarb, iprovalicarb, valiphenal, mandipropam id, oxytetracycline, methasulfocarb, fluopicolide, acibenzolar-S-methyl, probenazole, tiadinil, isotianil, ethaboxam, cyflufenam id, proquinazid, metrafenone, copper (different, salts), sulfur, ferbam, mancozeb, maneb, metiram, propineb, thiram, zineb, ziram, captan, captafol, folpet, chlorothalonil, dichlofluanid, tolylfluanid, dodine, guazatine, iminoctadine, anilazine, dithianon, mineral oils, organic oils, potassium bicarbonate, tridemorph anilinopyrimidines, antibiotics, cycloheximid, griseofulvin, dinitroconazole, etridazole, perfurazoate, 5-chloro-7-(4-methyl-piperidin-1-yl)-6-(2,4,6-trifluoro-phenyl)-[1,2,4]triazolo[1,5-a]pyrimidine, 2-butoxy-6-iodo-3-propyl-chromen-4-one, 3-(3-Bromo-6-fluoro-2-methyl-indole-1-sulfonyl)-[1,2,4]triazole-1-sulfonic acid dimethylamide, nabam, metam, polycarbamate, dazomet, 3-[5-(4-chloro-phenyl)-2,3-dimethyl-isoxazolidin-3-yl]-pyridine, Bordeaux mixture, copper acetate, copper hydroxide, copper oxychloride, basic copper sulfate, nitrophenyl derivatives, dinobuton, nitrophthalisopropyl phenylpyrroles, sulfur, sulfur organometallic compounds, phthalide, toloclofos-methyl, N-(2-{4-[3-(4-chloro-phenyl)-prop-2-ynyloxy]-3-methoxy-phenyl}-ethyl)-2-methanesulfonylamino-3-methyl-butyramide, N-(2-{4-[3-(4-chloro-phenyl)-prop-2-ynyloxy]-3-methoxy-phenyl}-ethyl)-2-ethanesulfonylamino-3-methyl-butyramide; 3,4-dichloro-isothiazole-5-carboxylic acid (2-cyano-phenyl)-amide, flubenthiavalicarb, 3-(4-chloro-phenyl)-3-(2-isopropoxycarbonylamino-3-methyl-butyrylamino)-propionic acid methyl ester, {2-chloro-5-[1-(6-methyl-pyridin-2-ylmethoxyimino)-ethyl]-benzyl}-carbamic acid methyl ester, {2-chloro-5-[1-(3-methyl-benzyioxyimino)-ethyl]-benzyl}-carbamic acid methyl ester, enestroburin, sulfenic acid derivatives, flumetover, cyflufenamid or (Z)-N-[α-(cyclopropylmethoxyimino)-2,3-difluoro-6-(trifluoromethyl) benzyl]-2-phenylacetamide, thiabendozole, and triflumizole.

Additionally, the container may comprise one or more antimicrobials, including but not limited to: bleach, Plant Preservative Mixture™, quaternary ammonium or pyridinium salts, the copper salt of cyanoethylated sorbitol (as described in U.S. Pat. No. 6,978,724), silver salts and silver nanoparticles can be used. Additionally, the container may comprise one or more antibiotics, including but not limited to: cefotaxime, carbenicillin, chloramphenicols, tetracycline, erythromycin, kanamycin, neomycin sulfate, streptomycin sulfate, gentamicin sulfate, ampicillin, penicillin, ticarcillin, polymyxin-B and rifampicin chlorhexidine, chlorhexidine acetate, chlorhexidine gluconate, chlorhexidine hydrochloride, chlorhexidine sulfate, hexamethylene biguanides, oligo-hexamethyl biguanides, silver acetate, silver benzoate, silver carbonate, silver chloride, silver iodate, silver iodide, silver lactate, silver laurate, silver nitrate, silver oxide, silver palmitate, silver protein, silver sulfadiazine, polymyxin, tetracycline, tobramycin, gentamicin, rifampician, bacitracin, neomycin, chloramphenical, miconazole, tolnaftate, oxolinic acid, norfloxacin, nalidix acid, pefloxacin, enoxacin, ciprofloxacin, ampicillin, amoxicillin, piracil, vancomycin, polyhexamethylene biguanide, polyhexamethylene biguanide hydrochloride, polyhexamethylene biguanide hydrobromide, polyhexamethylene biguanide borate, polyhexamethylene biguanide acetate, polyhexamethylene biguanide gluconate, polyhexamethylene biguanide sulfonate, polyhexamethylene biguanide maleate, polyhexamethylene biguanide ascorbate, polyhexamethylene biguanide stearate, polyhexamethylene biguanide tartrate, polyhexamethylene biguanide citrate and combinations thereof.

In order to prevent insect damage, the artificial seed may also comprise one or more insecticides. Examples of suitable pesticidal compounds include, but are not limited to, abamectin, cyanoimine, acetamiprid, nitromethylene, nitenpyram, clothianidin, dimethoate, dinotefuran, fipronil, lufenuron, flubendamide, pyripfoxyfen, thiacloprid, fluxofenime, imidacloprid, thiamethoxam, beta cyfluthrin, fenoxycarb, lamda cyhalothrin, diafenthiuron, pymetrozine, diazinon, disulphoton; profenofos, furathiocarb, cyromazin, cypermethrin, tau-fluvalinate, tefluthrin, chlorantraniliprole, cyantraniliprole, flonicamid, metaflumizone, spirotetramat, Bacillus thuringiensis products, azoxystrobin, acibenzolor s-methyl, bitertanol, carboxin, Cu₂O, cymoxanil, cyproconazole, cyprodinil, dichlofluamid, difenoconazole, diniconazole, epoxiconazole, fenpiclonil, fludioxonil, fluoxastrobin, fluquiconazole, flusilazole, flutriafol, furalaxyl, guazatin, hexaconazole, hymexazol, imazalil, imibenconazole, ipconazole, kresoxim-methyl, mancozeb, metalaxyl, R-metalaxyl, mefenoxam, metconazole, myclobutanil, oxadixyl, pefurazoate, paclobutrazole, penconazole, pencycuron, picoxystrobin, prochloraz, propiconazole, pyroquilone, SSF-109, spiroxamin, tebuconazole, thiabendazole, thiram, tolifluamide, triazoxide, triadimefon, triadimenol, trifloxystrobin, triflumizole, triticonazole, uniconazole.

The artificial seed may comprise other crop protection chemicals, including but not limited to nematicides, termiticides, molluscicides, miticides and acaricides.

In the process of artificial seed preparation and following addition of the regenerable plant tissue, and in some cases, the nutrient source, the opening in the container can be secured. The container can have more than one opening, for example, the container can have a top opening and a bottom opening. Depending on the design and method of planting, optionally one or both openings can be secured. Identical materials can be used as closures for the top opening and the bottom opening of the container. Alternatively, different materials can be used as closures for securing the opening(s). Suitable materials to be used as closures or for the container can include, for example, various types of paper, wax, PARAFILM®, pre-stretched PARAFILM®, biodegradable polymers including poly(lactide), poly(L-lactide), poly(D-lactide), poly(D,L-lactide), stereocomplexes of poly(L-lactide) with poly(D-lactide) and poly(hydroxyl alkanoate)s, natural and synthetic polymers including but not limited to, poly(styrene), poly(alkyl (meth)acrylates), poly(vinyl acetate),), poly(vinyl pyridine), polycarbonate, epoxy resins, alkyd resins, polyolefins, photodegradable polymers, polyesters, polyamides, natural rubber, polysaccharides including but not limited to cellulose, zein, crosslinked versions thereof, plasticized versions thereof, copolymers thereof and combinations thereof. In some embodiments, the closure or the container comprises, or alternatively consists of, a bilayer or a multilayer structure.

In some embodiments, the inner layer or layers of the closure or container consists of water insoluble substances which may also be moisture barriers. These layers can be penetrable by the growing regenerable plant tissue. The outer layer or layers are degradable and may be impenetrable by the regenerable plant tissue. In some embodiments, the outer layers serve to mechanically strengthen the artificial seed while being degradable, while the inner layers serve to protect the regenerable tissue from moisture loss while allowing it to escape at an appropriate growth stage. In some embodiments, the container comprises or consists of a 3-layer structure having an outer layer comprising the polylactic acid composition, a middle paper or cellulosic layer and an inner layer comprising the polylactic acid composition. In some embodiments, the container comprises or consists of a bilayer structure having the polylactic acid composition and a paper or cellulosic layer.

In some embodiments, when the closure comprises more than one layer, various parts of the closures for either the top opening or the bottom opening can be generated separately and then assembled together to make the final closure for the artificial seed. Film closures used for securing the top opening may be pre-stretched and provide a transparent closure for the top closure which allows the passage of light into the artificial seed container for the regenerable plant tissue in the container to grow. The bottom part of the closure can have additional layers such as a solid fat layer to prevent contact of the soil moisture with the bottom film closure.

The closure or one of the layers of a multilayer closure or container can contain oil. The oil suitable for the application can have the following characteristics: it should melt between 30° C. to 38° C. and be solid at room temperature (from about 20° C. to about 25° C.). Various types of oil and triglycerides (fat) can be used. Suitable oils can include, for example, butter, cocoa butter, palm oil, palm stearine, lard, vegetable oil, castor oil, soybean oil, rapeseed oil, mineral oil or a combination thereof. In an embodiment, vegetable oil shortening, e.g., CRISCO®, can be used. In another embodiment, the closure may be composed of an oil-gel. An oil-gel is defined as an oil that, through combination with one or more additives, does not flow over a finite range of temperature suitable for the application. In one embodiment, the oil-gel is formed by dissolving a compound in an oil at elevated temperature, and then cooling that solution to form a gel. Suitable oils include, but are not limited to, vegetable oil, castor oil, soybean oil, rapeseed oil, and mineral oil. Suitable oils can also include, for example, block polymers and associative, low molecular weight substances. Block polymers include, but are not limited to, styrenic block copolymers such as those sold under the trade name KRATON® (Kraton Polymers, Houston, Texas), block copolymers of ethylene oxide and propylene oxide, such as those sold under the name PLURONIC® (BASF, Ludwigshafen, Germany). Styrenic block copolymers include but are not limited to poly(styrene-b-isoprene-b-styrene), poly(styrene-b-butadiene-b-styrene) and hydrogenated versions thereof. Oil-gels suitable for this application will have mechanical properties weak enough to permit penetration by the growing regenerable plant tissue.

In some embodiments, the closure or container can comprise one or more layers of oil-gel and one or more layers of water soluble film. In this embodiment, the oil-gel layer prevents contact of the soil moisture with the bottom water soluble film layer. This preserves the structure of the artificial seed during storage. When the artificial seed is planted and irrigated, the water soluble film can dissolve, leaving behind the oil-gel, which allows the growth of the plant.

The closure or container can comprise one or more layers of paper. The paper suitable for application in preparation of the can have a variety of thicknesses or basis weights. Non-limiting examples include paper used for postcards, business cards, playing cards and scrapbooking paper. In an embodiment, card stock paper can be used. The multilayer can comprise a paper plastic laminate, such as those widely known to be used for paper beverage containers. The plastic layer comprises a polymer which is extruded onto the paper in a coating process. The plastic layer may comprise polymers such as polylactide, polylactide containing degradation promoting additives, polyolefins, polyester. The plastic layer can serve to reduce the water permeability of the paper plastic laminate and/or prevent or retard softening of the paper layer by water.

Other materials such as wax-impregnated cheese cloth, wax-impregnated paper can also be included in preparation of the closures.

The closures used for securing the bottom opening of the containers of the artificial seeds comprise one layer or more than one layer. The bottom closure can be just one layer of degradable or water soluble films at both top and bottom, or the bottom closure can have more than one layer including a paper disc under the film. Alternatively the bottom closure can have multiple layers comprising an oil or a fat layer, followed by an optional paper layer, followed by a film layer; whereas the top can still be the single film layer which is transparent to light. Finally the artificial seed's top and bottom closures could be made separately and then put together by inserting the top portion into the bottom like a capsule. In some embodiments, the bottom closure can consists of an oil or fat layer and an optional paper layer, followed by a film layer.

In an embodiment one of the layers of a multilayer structure for the container or closures can consist of a hydrophobic substance. A hydrophobic substance is defined as a substance that has a lower surface energy than water. Hydrophobic substances include but are not limited to oils, fats, greases, polyolefins, polyolefin oligomers, polylactide based compositions, triglycerides, polyethylene, polypropylene, ethylene propylene copolymers, polybutadiene, polyisoprene and polyisobutylene. In one embodiment, the hydrophobic substance can melt or flow at a temperature above 1° C. In one embodiment, the hydrophobic substance can melt or flow above 10° C. In one embodiment, the hydrophobic substance can melt or flow above 15° C. In one embodiment, the hydrophobic substance can melt or flow above 20° C. In another embodiment, the hydrophobic substance can melt or flow above 25° C. In another embodiment, the hydrophobic substance can melt or flow above 30° C. In another embodiment, the hydrophobic substance can melt or flow above 35° C. In another embodiment, the hydrophobic substance can melt or flow above 40° C. In another embodiment, the hydrophobic substance can melt or flow above 45° C. In another embodiment, the hydrophobic substance can melt or flow below 50° C.

In another embodiment, one of the layers of the multilayer structure for the container or closures can consist of a moisture barrier. A moisture barrier is defined as a substance that reduces or prevents the transport of water or water vapor. Moisture barriers include but are not limited to polyolefins, ethylene copolymers, polyesters, polyamides, polydienes, polylactide based compositions, polycarbonates, polyethers, polysulfides, polyim ides, polyanhydrides, polyurethanes, poly(vinyl esters), poly(vinyl ethers), natural polymers, block copolymers, crosslinked polymers, proteins and blends and crosslinked versions thereof.

In another embodiment, one of the layers of the multilayer closure or container can be degradable. Degradable materials include but are not limited to poly(lactic acid), amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(1,3-propanediol succinate), poly(propylene succinate), polyglycolide, poly(caprolactone), poly(butylene succinate), poly(ethylene succinate), poly(ethylene carbonate), poly(propylene carbonate), poly(butylene terephthalate adipate), poly(propylene terephthalate succinate), poly(propylene terephthalate adipate), poly(vinyl alcohol), cellulose acetate, cellulose butyrate acetate; and blends, copolymers or crosslinked versions thereof.

In some embodiments, the closure is made of degradable plastic materials such as poly(lactic acid), poly(hydroxybutyrate), poly(hydroxybutyrate-co-valerate), or blends thereof, optionally with starch, cellulose, chitosan and plasticizers, including but not limited to sorbitol, glycerol, and water. These blends may be formed by solution blending or melt blending. In some embodiments, the closure comprises a layer comprising the polylactic acid composition comprising polylactic acid, the degradable additives and optionally, the thermal stability additive.

In other embodiments, the closure comprises, or alternatively consists of, rapidly dissolvable blends of poly(vinyl alcohol) with starch, cellulose fibers and/or glycerol, optionally crosslinked, with a suitable agent, including but not limited to hexamethoxymethylmelamine or glutaraldehyde. This provides materials which are degradable in moist soil conditions, permitting rapid growth of the regenerable plant tissue inside. The starch may be from sources including but not limited to potato, corn, rice, wheat and cassava, and may be modified or unmodified. Additional additives may include, but are not limited to poly(ethylene glycol), citric acid, urea, water, salts including but not limited to sodium acetate, potassium nitrate and ammonium nitrate, fertilizers, agar, xanthan gum, alginate, cellulose derivatives including but not limited to hydroxypropylcellulose, methylcellulose and carboxymethylcellulose.

In some embodiments, the container may have a top and a bottom opening which can be secured. In an embodiment, pre-stretched PARAFILM® F can be used to secure both the top opening and the bottom opening of the container. In another embodiment, the closure for the openings comprise, or alternatively consist of, alkyd resin films. Such alkyd resins are well known in the art, and can be formed through the reaction of unsaturated vegetable oils with polyols and optionally cured with metal catalysts. Suitable alkyd resins include, but are not limited to BECKOSOL® 11-035 and AMBERLAC® 1074 (Reichhold Corp, Durham, N.C.).

In another embodiment, the closure for the openings comprises, or alternatively consists of, block copolymers. Block copolymers include two or more segments of chemically distinct constitutional repeating units, linked covalently. These block copolymers may be biodegradable. In one embodiment, polyester block copolymers are used. Such polymers may be elastomeric, allowing the regenerable plant tissue to puncture them easily. The block copolymers contain blocks including but not limited to: poly(lactic acid), poly(lactide), poly(L-lactic acid), poly(D-lactic acid), poly(D,L-lactic acid), poly(caprolactone), poly(caprolactone-co-lactic acid), poly(dimethylsiloxane), poly(vinyl alcohol), poly(vinyl acetate), poly(ethylene glycol), poly(propylene glycol), poly(carbonate)s, polyethers, polyesters. In one embodiment, the block copolymers can consist of poly(L-lactic acid-b-caprolactone-co-D,L-lactic acid-b-L-lactic acid). In another embodiment, the block copolymer consists of poly(D,L-lactic acid-b-dimethyl siloxane-b-D,L-lactic acid).

In other embodiments, the openings can be secured using porous materials, including but not limited to, a crimp, a fold, a flap, a porous material, a mesh, a screen, cotton, gauze or a staple.

Alternatively, the top and bottom openings can be secured by folding, crimping, pinching, stapling, or fastening the opposing sides of the container together. In one embodiment, the bottom opening can be secured by stapling its sides together.

In some embodiments, the openings can be secured by the flap-like structures, wherein one or more flexible flaps protrude over the opening. The flaps are flexible enough to allow the plantlet to push them apart as it grows. In other embodiments, the flaps form a slotted lid or “flower” or “blossom”-shaped lid.

In another embodiment, the container can have one or more openings on the side of the container. These side openings can be in addition to the top and bottom openings. Alternatively, the container can have only side openings without top or bottom openings. These openings can also be secured using methods and materials described above.

In another embodiment, the container can possess anchoring devices. Such devices include, but are not limited to flaps, barbs, stakes and ribs. The anchoring devices can be foldable or collapsed, to reduce space prior to planting. In such cases, a restraint may be used to hold the anchoring device in a folded or collapsed state. Such restraints may include, but are not limited to tapes, bands, and adhesives.

Following the assembly of the container, adding the the regenerable plant tissue, the nutrient source, if required, and securing the top opening and the bottom opening, the artificial seeds thus created, can be planted in soil. Any kind of soil such as field soil, sandy soil, silty soil, clay soil, organic rich soil, organic poor soil, high pH soil, low pH soil, loam, synthetic soil, vermiculite, potting soil, nursery soil, topsoil, mushroom soil and sterilized versions thereof can be used for this purpose. In an embodiment, Metro-Mix® 360 (and field soil—such as that from farms or other natural sources around the world) can be used for planting the regenerable plant tissue in the containers. The artificial seeds will then sprout or germinate at some frequency thereafter. As used herein, “sprouting” and “germination” mean the protrusion of the regenerable plant tissue from the boundaries of the container of the artificial seed due to growth of the regenerable plant tissue.

The artificial seeds described herein are suited for storage prior to planting. Storage conditions may include, but are not limited to ambient temperature, refrigerated temperature, sub-ambient temperature, sub-ambient oxygen concentration, sub-ambient illumination, in light or in darkness, in external packaging, under air or in an inert atmosphere. Sub-ambient temperature is defined herein as temperature below the ambient temperature. Sub-ambient illumination is defined herein as illumination levels below the ambient illumination. Sub-ambient oxygen is defined as levels of oxygen below that present in the natural atmosphere. The storage duration may be as long as one year, or a few months, but may also be on the order of weeks or days.

In some embodiments, holes, cuts, breaches or slits may be made in the artificial seed at the time of planting in order to facilitate the growth of the regenerable plant tissue. This can enable the shoots or the roots to grow out of and escape the container.

The present disclosure provides for production of artificial seeds that can develop into fully grown crops for propagation in the field. For example, the artificial seed can provide for an economical method of propagating hard-to-scale up plants such as sugarcane that can allow their rapid propagation to meet the growing global demand for sugarcane production. Also, the artificial seed can provide for a simpler, safer and more economical planting method compared to the traditional planting of sugarcane stalks and billets via either mechanical or manual means. Simply reducing the weight and volume of planting material, from sugarcane stalks and billets to artificial seeds, can save the energy and time required to transport planting materials to the field for planting.

The present disclosure also relates to a method of forming an article. The method comprises the steps of:
A) heating a polylactic acid composition above the softening point of the polylactic acid composition;
B) forming the softened composition into an article; and
C) cooling the formed article.
   Wherein the polylactic acid composition comprises polylactic acid, at least two degradation additive and optionally, the thermal stability additive, and wherein the softening temperature of the cooled article is in the range of from 55° C. to 160° C. In other embodiments, the article is an artificial seed or the container of an artificial seed.

In some embodiments, the method of forming the softened composition can be an extrusion step or a thermoforming step. Each of the extrusion or thermoforming steps is well known in the art.

The above description of various illustrated embodiments are not intended to be exhaustive or to limit the precise form of the artificial seed. While specific embodiments of, and examples, are described herein for illustrative purposes, various equivalent modifications are possible within the scope of the disclosure, as those skilled in the relevant art will recognize. The teachings provided herein can be applied to other purposes, other than the examples described above. Numerous modifications and variations are possible in light of the above teachings and, therefore, are within the scope of the appended claims.

These and other changes may be made in light of the above detailed description. In general, in the following claims, the terms used should not be construed to limit the specific embodiments disclosed in the specification and the claims.

Certain teachings related to viable plant artificial seeds were disclosed in U.S. Provisional patent application No. 61/578,432, filed Dec. 21, 2011, the disclosure of which is herein incorporated by reference in its entirety.

The entire disclosure of each document cited (including patents, patent applications, journal articles, abstracts, manuals, books, or other disclosures) is herein incorporated by reference in their entireties.

The following examples are not intended to limit the scope of the disclosure. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; temperature is in degrees centigrade; and pressure is at or near atmospheric.

### EXAMPLES

Unless otherwise noted, all ingredients are available from the Sigma Aldrich Company, St. Louis, Mo.

INGEO® 2003D polylactic acid is available from the Natureworks, LLC., Minnetonka, Minn.

Iron (III) stearate was obtained from TCI Chemicals, Portland, Oregon.

IRGANOX® 1010 was obtained from BASF (Ludwigshafen, Germany).

METRO-MIX® 360 growing medium is available from Sun Gro Horticulture, Agawam, Mass.

Poly(lactic acid) pellets were used as received (factory dried in sealed bags) or otherwise dried under partial vacuum with nitrogen purging at 80-100° C. for 14 hours. Oleic acid was technical grade, 90% purity from Aldrich (product #364525). Corn starch (Aldrich product S4126) was dried under vacuum with nitrogen purging at 110° C. for 1 to 3 days. The poly(lactic acid) pellets were compounded in a twin screw extruder with the additives in the concentrations listed in Table 1 at temperatures ranging from 180-220° C. The compounded pellets were then dried at 40-50° C. under partial vacuum with nitrogen purging for 24 hours. The pellets were then extruded into approximately 5 mil thick films using a single screw extruder and a coat-hanger die. The amounts in Table 1 are all in percent by weight based on the total amount of the polymer composition.

TABLE 1

| | | degradation | degradation | degradation |
|---|---|---|---|---|
| Composition | PLA | additive 1 | additive 2 | additive 3 |
| 1 | INGEO ® 2003D | 28.9% corn | 3.6% oleic acid | — |
| | | starch | | |
| 2 | INGEO ® 2003D | 29.8% corn | 0.7% iron (III) | — |
| | | starch | stearate | |
| 3 | INGEO ® 2003D | 28.7% corn | 0.7% iron (III) | 3.5% oleic acid |
| | | starch | stearate | |
| Comparative A | INGEO ® 2003D | 0.5% iron | 5% oleic acid | — |
| | | stearate | | |
| Comparative B | INGEO ® 2003D | 30% corn | — | — |
| | | starch | | |
| Comparative C | INGEO ® 2003D | 20% corn | — | — |
| | | starch | | |
| Comparative D | INGEO ® 2003D | 5.0% oleic acid | | |

*Composition A also included 0.1% of IRGANOX ® 1010.

Soil Degradation of Polymer Films

Polymer films from Table 1 were cut into 2″×2″ pieces. The film thickness was approximately 127 um. Fifteen films were cut from each polymer blend, numbered, and the initial weight was recorded. A cross-mesh bag (4″×5″, Item #09-212-34, PaperMart) was filled with native soil from the fields at Stine Haskell Research Center, Newark, Del. One polymer film was placed into the soil in the bag. The bag was then buried under 2″ of soil in the field. Three bags were removed from the field per polymer blend at 1, 2, 3, 4, and 5 months. The contents of the bag were carefully poured into a metal sieve (VWR, #14). The soil was washed off the polymer film and the film was dried at room temperature for 48 hours. The film was weighed and the percent weight loss as a function of initial weight was calculated. The Control example was a film of INGEO® 2003D with no additives. The results of soil degradation study on polymer films are shown in Table 2. Standard deviation of the weight loss values are given in parentheses.

TABLE 2

| Composition | 1 month | 2 months | 3 months | 4 months | 5 months |
|---|---|---|---|---|---|
| 1 | 30.22% | 55.42% | 66.70% | 49.36% | 85.98% |
| | (7.11%) | (13.35%) | (13.35%) | (7.80%) | (16.26%) |
| 2 | 12.95% | 12.68% | 26.72% | 22.99% | 52.74% |
| | (7.55%) | (7.16%) | (15.41%) | (7.00%) | (51.40%) |
| 3 | 36.05% | 71.45% | 70.72% | 63.90% | 65.97% |
| | (7.77%) | (3.53%) | (23.08%) | (7.73%) | (2.07%) |
| Control | −0.27% | 0.00% | 0.00% | −0.08% | −0.82% |
| | (0.04%) | (0.00%) | (0.58%) | (0.14%) | (0.50%) |
| Comparative A | −9.26% | −3.77% | −1.51% | −1.31% | −1.98% |
| | (5.98%) | (6.16%) | (0.60%) | (0.24%) | (2.37%) |
| Comparative B | 11.14% | 29.41% | 11.36% | 33.11% | n/a |
| | (2.87%) | (17.51%) | (0.81%) | (3.29%) | |
| Comparative C | 1.06% | 1.94% | 3.00% | 3.25% | 5.09% |
| | (0.92%) | (0.19%) | (1.79%) | (1.08%) | (2.49%) |
| Comparative D | 0.15% | −1.73% | −1.39% | −1.45% | −0.47% |
| | (0.68%) | (1.23%) | (0.34%) | (0.77%) | (1.20%) |

The examples in Table 2 show that polylactic acid compositions having two or more degradation additives (1-3) exhibit greater degradation rate than compositions containing only 1 degradation promoting additive (comparative B-D). Comparative composition A contains degradation promoting additives aimed at both photodegradation (iron III stearate) and hydrolytic degradation (oleic acid). The lack of two degradation additives aimed at hydrolytic degradation results in this composition not degrading quickly in soil.

What is claimed is:

1. An artificial seed comprising one or more regenerable plant tissues, a container, an airspace, a nutrient source and an additional feature, wherein the additional feature is:
a) a penetrable or degradable region through which the regenerable plant tissue grows;
b) a monolayer portion of the container;
c) a region of the container that flows or creeps between 25° C. and 50° C.;
d) a separable closure which is physically displaced during regenerable plant tissue growth;
e) one or more openings in the sides or bottom of the container;
f) a conical or tapered region leading to an opening less than 2 centimeters wide at the apex and wherein the angle of the conical or tapered region is less than 135 degrees measured from the opposite sides;
g) a plurality of flexible flaps through which the regenerable tissue grows; or
h) a combination thereof; and
wherein the container comprises a degradable portion comprising a polylactic acid composition and wherein the polylactic acid composition comprises polylactic acid, at least two degradation additives and optionally, a thermal stability additive.

2. The artificial seed of claim 1 wherein the degradation additive is a combination of at least two of C8 to C18 carboxylic acid, poly(meth)acrylic acid, anhydrides, maleic anhydride containing polymers, polyvinyl alcohol, polyvinyl pyrrolidone, starch, iron powder, iron (III) carboxylate salt, cobalt carboxylate salt, manganese carboxylate salt, polyethylene glycol, clinoptilolite, zeolite, gypsum, diatomaceous earth, calcium phosphate, calcium carbonate, keratin, silica, alumina, clay, cloisite, montmorillonite or a combination thereof.

3. The artificial seed of claim 1 or 2 wherein the thermal stability additive is calcium carbonate, a nucleating agent, a reinforcing fiber, glass fiber, nanocellulose, mica, carbon fiber, sisal, wollastonite, sepiolite, a layered mineral, clay or a combination thereof.

4. The artificial seed of any one of claims 1 to 3 wherein the nutrient source further comprises soil, coconut coir, vermiculite, an artificial growth medium, agar, a superabsorbent polymer, a plant growth regulator, a plant hormone, micronutrients, macronutrients, water, fertilizer, peat or a combination thereof.

5. The artificial seed of any one of claims 1 to 4 wherein the regenerable plant tissue is sugar cane, a gram inaceous plant, saccharum spp, saccharum spp hybrids, miscanthus, switchgrass, energycane, sterile grasses, bamboo, cassava, corn, rice, banana, potato, sweet potato, yam, pineapple, trees, willow, poplar, mulberry, ficus spp, oil palm, date palm, poaceae, verbena, vanilla, tea, hops, Erianthus spp, intergeneric hybrids of Saccharum, Erianthus and Sorghum spp, African violet, apple, date, fig, guava, mango, maple, plum, pomegranate, papaya, avocado, blackberries, garden strawberry, grapes, canna, cannabis, citrus, lemon, orange, grapefruit, tangerine or dayap.

6. The artificial seed of any one of claims 1 to 5, further comprising a cylindrical tube with a conical top, a two-part tube with a porous bottom section and a nonporous top section, a flexible packet, a semi-flexible packet, one or more rigidifying elements, a multicomponent extruded tube, a rolled tube structure capable of unravelling, an anchoring device, a two-part tube with a hinged edge, a two-part tube held together with adhesive, a tubular shape, a container portion in contact with soil that degrades faster than the portion above the soil, an airspace comprising multiple compartments, a closed bottom end that retains moisture, a cap attached by an adhesive joint or a cap attached by insertion into the container.

7. The artificial seed of any one of claims 1 to 6 wherein the container further comprises a transparent material, a translucent material, an opaque material, a porous material, a nonporous material, a permeable material, an impermeable material or a combination thereof.

8. The artificial seed of any one of claims 1 to 7 wherein one or more of the openings are secured using a crimp, a fold, a flap, a porous material, a mesh, a screen, cotton, gauze or a staple.

9. The artificial seed of any one of claims 1 to 8 further comprising a fungicide, a nematicide, an insecticide, an antimicrobial compound, an antibiotic, a biocide, an herbicide, a plant growth regulator, a plant growth stimulator, a microbe, a molluscicide, a miticide, an acaricide, a bird repellant, an insect repellant, a plant hormone, a rodent repellant, fertilizers, hydrogels, superabsorbent polymers, fillers, water or a combination thereof.

10. The artificial seed of any one of claims 1 to 9 wherein the degradable portion comprises in the range of from 50 to 99 percent polylactic acid, in the range of from 0.1 to 50 percent by weight of the degradation additives and optionally, in the range of from 0.1 to 40 percent by weight of the thermal stability additive, wherein all percentages by weight are based on the total weight of the polylactic acid composition.

11. A polylactic acid composition comprising:
1) polylactic acid;
2) at least two degradation additives; and optionally,
3) a thermal stability additive.

12. The polylactic acid composition of claim 11 wherein the degradation additives are starch, a C8 to C18 carboxylic acid, iron, an iron (III) carboxylate salt, polyethylene glycol, poly(ethylene oxide), clinoptilolite, gypsum, diatomaceous earth or a combination thereof.

13. The polylactic acid composition of claim 11 or 12 wherein the optional thermal stability additive is a nucleating agent, a reinforcing fiber, a layered mineral clay, clay, sepiolite or a combination thereof.

14. The polylactic acid composition of any one claims 11 to 13 wherein the C8 to C18 carboxylic acid is decanoic acid, oleic acid, stearic acid or a combination thereof.

15. The polylactic acid composition of any one of claims 11 to 14 wherein the composition comprises:
1) 50 to 99 percent by weight of polylactic acid;
2) 0.1 to 50 percent by weight of at least two degradation additives; and optionally,
3) 0.1 to 40 percent by weight of the thermal stability additive,
wherein all of the percentages by weight are based on the total amount of the polylactic acid composition.

16. A method comprising the steps of:
A) heating the polylactic acid composition of claim 11 above the softening point of the polylactic acid composition;
B) forming the softened composition into an article; and
C) cooling the formed article,
wherein the softening temperature of the cooled article is in the range of from 55° C. to 160° C.

17. The method of claim 16 wherein the article is an artificial seed.

18. The method of claim 16 or 17 wherein the step of forming is an extruding step or a thermoforming step.

19. The method of any one of claims 16 to 18 wherein the thermal history of the article allows sufficient crystallization of the polylactic acid composition to provide a softening temperature in the range of from 55° C. to 160° C.
